(19) United States
(12) Patent Application Publication
(10) Pub. No.: US 2026/0051100 A1
(43) Pub. Date: Feb. 19, 2026

(54) IMAGE RECONSTRUCTION FOR MAGNETIC RESONANCE IMAGING

(71) Applicant: Hyperfine Operations, Inc., Guilford, CT (US)

(72) Inventor: Jo Schlemper, Long Island City, NY (US)

(73) Assignee: Hyperfine Operations, Inc., Guilford, CT (US)

(21) Appl. No.: 19/370,696

(22) Filed: Oct. 27, 2025

Related U.S. Application Data
(63) Continuation of application No. PCT/US2024/026663, filed on Apr. 26, 2024.
(60) Provisional application No. 63/499,009, filed on Apr. 28, 2023.

(51) Int. Cl. G06T 11/00; A61B 5/00; A61B 5/055

(52) U.S. Cl. CPC G06T 11/006; A61B 5/055; A61B 5/7267; G06T 11/008; G06T 2211/441

(57) ABSTRACT

Systems and methods for training a machine-learning model to generate denoised and dealiased image data are provided. The present disclosure provides techniques for training a machine-learning (ML) model to generate denoised and dealiased imaging data. A method includes (1) training a first ML model using a first training dataset comprising first image data to obtain a second ML model; and (2) training (a) the second ML model or (b) a third ML model using a second training dataset to obtain a fourth ML model. The second training dataset includes (i) the first image data and (ii) training image data obtained by applying at least one of the second ML model or the third ML model to second image data. The denoising and dealiasing ML model may be either the fourth ML model or derived from the fourth ML model.

### CROSS-REFERENCE TO RELATED APPLICATIONS

This application claims the benefit under 35 U.S.C. § 120 as a continuation of International Patent Application No. PCT/US2024/026663 filed Apr. 26, 2024, which claims priority to U.S. Provisional Patent Application No. 63/499,009 filed Apr. 28, 2023, the contents of which are incorporated herein by reference in their entirety for all purposes.

### TECHNICAL FIELD

This application relates generally to reducing noise in medical imaging through machine learning, for such applications as magnetic resonance imaging (MRI) that may use, for example, low-field MRI systems. Example machine learning approaches include a two-step semi-supervised error-correcting and/or artifact-correcting (e.g., denoising and/or dealiasing) approach to reduce noise and/or reduce artifacts in low-field diffusion magnetic resonance (MR) images.

### BACKGROUND

Magnetic resonance imaging (MRI) systems may be utilized to generate images of the inside of the human body. MRI systems may be used to detect magnetic resonance (MR) signals in response to applied electromagnetic fields. The MR signals produced by MRI systems may be processed to produce images, which may enable observation of internal anatomy for diagnostic or research purposes. It may be challenging to accurately reconstruct MR signals captured by MRI systems while removing enough noise such that anatomical structures are sufficiently observable.

### SUMMARY

At least one aspect of the present disclosure is directed to a method of generating a trained machine-learning (ML) model for image reconstruction. Generating the trained ML model may comprise: (1) using a first training dataset to update a first ML model to obtain a second ML model, the first training dataset comprising first image data; and (2) using a second training dataset to update the second ML model to obtain the trained ML model. The second training dataset may comprise the first image data and training image data. The training image data may be obtained by applying the second ML model to second image data.

In various embodiments, at least one of the first training dataset or the second training dataset comprises simulated imaging data. In various embodiments, the simulated imaging data is based on simulated images of arbitrary contrast. In various embodiments, the second image data comprises non-independent and non-identically distributed noise. In various embodiments, the method comprises applying the trained ML model to a patient image to obtain a reconstructed patient image. In various embodiments, the patient image is acquired using at least one of a low-field magnetic resonance (MR) imaging system or a point-of-care (POC) MR imaging system. In various embodiments, the first image data and the second image data belong to separate domains. In various embodiments, the method comprises augmenting the training image data based on an augmentation process before step (2). In various embodiments, the second trained ML model comprises a plurality of convolutional neural network (CNN) layers. In various embodiments, the method further comprises generating the first training dataset by applying raw imaging data to an image reconstruction pipeline. In various embodiments, the method comprises adding simulated image corruption to the raw imaging data.

In another aspect, the disclosure is directed to a method comprising acquiring a patient image using an imaging system, and applying a trained machine-learning (ML) model to the patient image to obtain a reconstructed patient image. The trained ML model may have been generated by any of the above methods.

In various embodiments, the patient image is acquired using at least one of a low-field MR imaging system or a POC MR imaging system.

In yet another aspect, the disclosure is directed to a non-transitory computer-readable storage medium comprising instructions that, when executed by one or more processors of a computing system, cause the computing system to: acquire patient image data using an imaging system; and obtain a reconstructed patient image based on the patient image data, wherein obtaining the reconstructed patient image comprises applying a trained machine-learning (ML) model to the patient image data. The trained ML model having been generated by any of the above methods.

In yet another aspect, the disclosure relates to a system comprising an imaging system configured to generate imaging data, and one or more processors configured to cause the imaging system to generate patient images, and apply a trained ML model to the patient images to generate reconstructed patient images. The trained ML model may have been generated by any of the above methods.

Training the ML model can be performed on one computing system, or using multiple computing systems that are in communication with each other (e.g., via one or more wired or wireless networks). Training of the ML model may be performed on one or more computing systems, and a trained ML model subsequently transmitted to one or more other systems for use. The one or more other systems may be, or may comprise, an MRI system and/or one or more computing systems in communication with the MRI system and/or with each other. In some embodiments, all process steps of using a trained ML model may be performed by the MRI system, while in other embodiments, some or all process steps of using the trained ML model may be performed by computing systems other than the MRI system. In an example, an MRI system may provide a first set of imaging data to a separate networked computing system, which will input the first set of imaging data (or a derivation of the first set of imaging data) to the trained ML model, and provide a second set of imaging data (e.g., an output of the trained ML model, or a derivation of the output) back to the MRI system.

At least one other aspect of the present disclosure is directed to a method for training a denoising and dealiasing machine-learning (ML) model to generate denoised (or “de-noised”) and/or dealiased (or “de-aliased”) image data. The method includes (1) training a first ML model using a first training dataset comprising first image data to obtain a second ML model; and (2) training (a) the second ML model or (b) a third ML model using a second training dataset to obtain a fourth ML model. The second training dataset includes (i) the first image data and (ii) training image data obtained by applying at least one of the second ML model or the third ML model to second image data. The denoising and dealiasing ML model may be either the fourth ML model or derived from the fourth ML model.

In some implementations, at least one of step (1) or step (2) comprises a supervised training process. In some implementations, in step (2) the fourth ML model is obtained by using the second training dataset to train the third ML model, and the third ML model has an architecture that differs from that of the second ML model. In some implementations, the second image data comprises non-independent and non-identically distributed noise. In some implementations, the method may include applying the denoising and dealiasing ML model to a patient image to obtain a denoised patient image.

In some implementations, the patient image is acquired using at least one of a low-field MR imaging system or a point-of-care (POC) MR imaging system. In some implementations, the first image data and the second image data belong to separate domains. In some implementations, the method may include augmenting the training image data based on an augmentation process before step (2). In some implementations, the second trained ML model comprises a plurality of convolutional neural network (CNN) layers.

In some implementations, the method may include generating the first training dataset by applying raw imaging data to an image reconstruction pipeline. In some implementations, the method may include adding simulated image corruption to the raw imaging data. In some implementations, the third ML model is derived from the second ML model.

At least one another aspect of the present disclosure is directed to a method comprising acquiring a patient image using an imaging system, and applying a denoising and dealiasing machine-learning (ML) model to the patient image to obtain a denoised patient image. The denoising and dealiasing ML model may be obtained by (1) training a first ML model using a first training dataset comprising first image data to obtain a second ML model; and (2) training (a) the second ML model or (b) a third ML model using a second training dataset to obtain a fourth ML model. The second training dataset includes (i) the first image data and (ii) training image data obtained by applying at least one of the second ML model or the third ML model to second image data. The denoising and dealiasing ML model may be either the fourth ML model or derived from the fourth ML model.

In some implementations, the patient image may be acquired using at least one of a low-field MR imaging system or a POC MR imaging system.

Yet another aspect of the present disclosure is directed to a system comprising an imaging system configured to generate imaging data, and one or more processors configured to cause the imaging system to generate patient images, and apply a denoising and dealiasing ML model to the patient images to generate denoised patient images. The denoising and dealiasing ML model may be obtained by using a first training dataset comprising first image data to obtain a first ML model; and using a second training dataset to obtain a second ML model. The second training dataset includes (i) the first image data and (ii) training image data obtained by applying at least one of the first ML model or a third ML model to second image data. The denoising and dealiasing ML model may be either the second ML model or derived from the second ML model.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification. Aspects may be combined and it will be readily appreciated that features described in the context of one aspect of the present disclosure may be combined with other aspects. Aspects may be implemented in any convenient form. In a non-limiting example, by appropriate computer programs, which may be carried on appropriate carrier media (computer readable media), which may be tangible carrier media (e.g. disks) or intangible carrier media (e.g. communications signals). Aspects may also be implemented using suitable apparatus, which may take the form of programmable computers running computer programs arranged to implement the aspect. As used in the specification and in the claims, the singular form of “a,” “an,” and “the” include plural referents unless the context clearly dictates otherwise.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1A illustrates example components of a magnetic resonance imaging system, in accordance with one or more implementations;

FIG. 1B illustrates an example system for training a denoising and dealiasing machine-learning model to generate denoised image data, in accordance with one or more implementations;

FIG. 2 depicts an example dataflow diagram of an example magnetic resonance image reconstruction pipeline, in accordance with one or more implementations;

FIG. 3 depicts an example dataflow diagram of a first stage of a training process for training a denoising and dealiasing machine-learning model to generate denoised image data, in accordance with one or more implementations;

FIG. 4 depicts an example dataflow diagram of a second stage of the training process for training a denoising and dealiasing machine-learning model to generate denoised image data, in accordance with one or more implementations;

FIG. 5 depicts a flowchart of an example method of training a denoising and dealiasing machine-learning model to generate denoised image data, in accordance with one or more implementations;

FIGS. 6A and 6B show example experimental data comparing approaches to denoise magnetic resonance images, in accordance with one or more implementations; and

FIG. 7 is a block diagram of an example computing system suitable for use in the various arrangements described herein, in accordance with one or more example implementations.

### DETAILED DESCRIPTION

Below are detailed descriptions of various concepts related to and implementations of techniques, approaches, methods, apparatuses, and systems for training a machine-learning model to generate image data that is denoised (e.g., with removed or reduced noise) and/or dealiased (e.g., aliasing artifacts removed or reduced), or that is otherwise reconstructed (e.g., for error-correction and/or artifact-correction. The various concepts introduced above and discussed in detail below may be implemented in any of numerous ways, as the described concepts are not limited to any particular manner of implementation. Examples of specific implementations and applications are provided primarily for illustrative purposes.

Magnetic resonance imaging (MRI) systems generate images for health evaluation. MRI images are generated by “scanning” a patient while the MRI system applies magnetic fields to the patient and particular data is captured. MRI scans produce raw scan data that can be transformed or otherwise processed into an image that can then be analyzed or reviewed to better evaluate a patient's health. MRI scans that take longer generally can capture more raw data that may be used to produce images, while faster MRI scans, which require patients to be in an MRI system for significantly less time, can produce images from less raw scan data. To allow for faster scans with high image quality, the MRI data is processed differently.

Not all data from imaging systems is usable, and such unusable data is referred to as “noise” that, if not taken into account, can be misleading when interpreting images. An important consideration in processing medical images is to increase the ratio of the usable data (the “signal” being sought) relative to the noise that is in the data. This is referred to as the “signal-to-noise ratio,” normally shortened to SNR. Because faster scans produce less raw data, ensuring that the “signal” fraction of that data is high relative to the “noise” fraction allows fast scans to be more revealing.

Faster scans may employ weaker magnetic field intensity. In clinical low-field MRI, magnetic resonance (MR) sequences are designed such that a reasonable SNR may be achieved within an acceptable scan time. Faster MRI scans, although advantageously requiring patients to be in an MRI system for significantly less time, may produce images from less raw scan data but with a relatively lower SNR when compared to longer, high-field MRI scans. Image denoising and dealiasing techniques may be utilized to further improve the SNR for scans captured using fast, low-field MRI systems. Increasing the SNR improves the accuracy of various downstream processing tasks, and may enable further reductions in scan time for low-field MR systems.

Machine-learning can be used to teach a computer to perform tasks, such as transforming raw scan data into images and reducing noise (“denoising” or “de-noising”), without having to specifically program the computer to perform those tasks. This is especially useful when, for example, images are to be constructed from fast raw scan data that can vary greatly from one patient to the next. This provides a machine-learning model that has learned to perform the particular task, but the effectiveness of the model in different situations can vary greatly depending on how the model was trained (or “taught”) to perform the task. One machine-learning approach is referred to as “deep learning” and is based on multiple layers or stages of artificial neural networks.

Deep learning based image denoising and dealiasing methods, although capable of succeeding at a variety of image denoising and dealiasing tasks, typically require a sufficiently large dataset of “clean” images (i.e., low-noise or denoised images) for training. These are difficult or otherwise impracticable to obtain for MRI in clinical settings. Unsupervised denoising and dealiasing may be used in such cases to train deep learning-based denoising and dealiasing models without requiring clean data. Such approaches may require the noise to be independent and identically distributed (i.i.d.) in the image. In contrast, the noise distribution in the images obtained through complex MR reconstruction pipelines may be non-i.i.d., and therefore incompatible with such approaches.

To address these shortcomings, the technical solution disclosed here may employ a two-stage process for training a denoising and dealiasing machine-learning model to generate denoised image data. The techniques described herein may effectively remove correlated MR noise without requiring clean images from the target domain (e.g., the domain of clinical MR images captured from low-field MR systems). In the first training step, a supervised training process may be performed to train a denoising and dealiasing machine-learning model (e.g., a denoising and dealiasing convolutional neural network (DNCNN), etc.) using a training set from a source domain (e.g., a domain of MR images captured using high-field MRI and having clean reference images, etc.).

Once the denoising and dealiasing machine-learning model is trained, it may be applied to various images captured from a target domain (e.g., low-field MR images which are not necessarily associated with clean reference images). The outputs of the denoising and dealiasing machine-learning model when executed over the images from the target domain are subjected to data augmentation (including, e.g., image sharpening, affine transformation, elastic deformation, inserting or adding different geometric objects, intensity augmentation, etc., or any combination thereof) to increase the size of the dataset, which is then included as part of a second training set to re-train the denoising and dealiasing machine-learning model. The denoising and dealiasing machine-learning model may then be re-trained using supervised learning approaches using the second training set.

The techniques described herein may be scaled to challenging clinical MRI reconstruction on portable low-field (e.g., that is less than about 0.5 T, that is less than about 0.2 T, that is between about 100 mT and about 400 mT, that is between about 200 mT and about 300 mT, that is between about 1 mT and 100 mT, that is between about 50 mT and about 100 mT, that is between about 40 mT and about 80 mT, that is about 64 mT, etc.) MRI systems, while demonstrating improved perceptual quality as compared to traditional denoising and dealiasing approaches. The advantages of the techniques described herein include the ability to train denoising and dealiasing machine-learning models using data that includes correlated noise but does not include clean reference images. The techniques described herein may provide competitive performance compared to unsupervised approaches and is robust across different noise levels. The systems and methods described herein therefore provide technical improvements over conventional MRI image denoising and dealiasing approaches.

In various embodiments, generating arbitrary contrast of MR images significantly increases the diversity of the contrast seen by the neural network during training. The network may be extended from two-dimensional (2D) reconstruction to multislice (or “multi-slice”) reconstruction. For example, multiple adjacent slices of MR frequency data may be used to predict the reconstruction simultaneously. In addition, the network may employ conjugate gradient descent to enhance data consistency. Example embodiments extend this for non-cartesian data, incorporating sample density compensation (SDC) and spectral normalization (SN) for low-field MR data. In some embodiments, both SDC and SN are components that help enhance enforcement of data consistency.

In various embodiments, MR reconstruction is enhanced by making the machine learning model operate more robustly on different input data. For example, in various embodiments, adding simulation increases the diversity of features seen by the network, hence increasing robustness. By extending the network to 2.5D (multislice), this approach increases the quality and robustness of the reconstruction. By using conjugate gradient descent for data consistency, for example, the fidelity (e.g., how appropriately the acquired data is represented) of the reconstruction is improved.

In various embodiments, a trained denoising network is applied to diffusion weighted imaging (DWI). For T2-weighted and DWI, example embodiments employ ensembling to combine outputs of multiple models (e.g., by taking an average, mean, or other statistic of the model outputs). This allows for reduction in errors of each model, resulting in improved image quality. An example network architecture uses multislice denoising by, for example, taking three adjacent slices (or, e.g., five or seven adjacent slices) of a noisy MR image and predicting the denoised instance of the middle slice. The disclosed approach reduces noise level in the image to improve image quality for, for example, T2 and DWI.

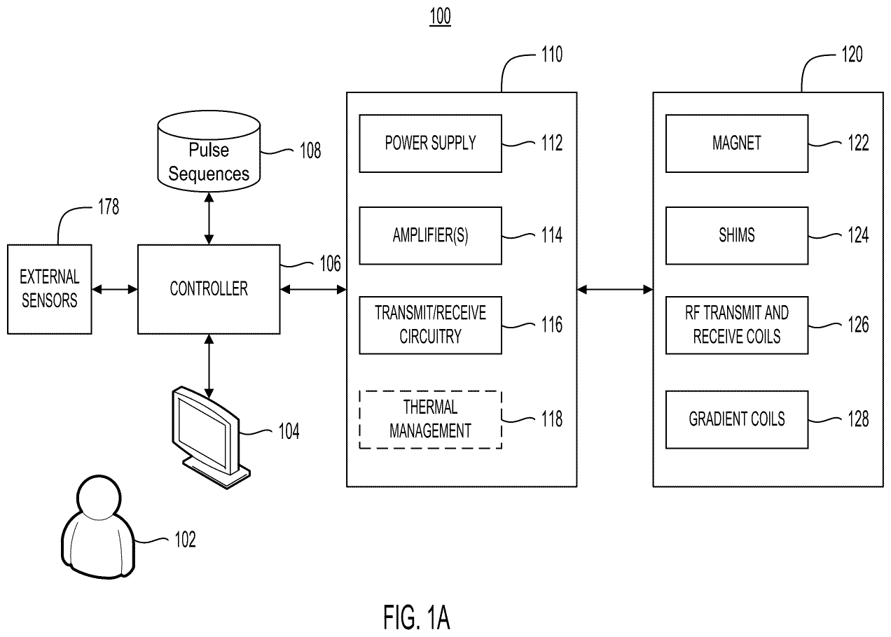

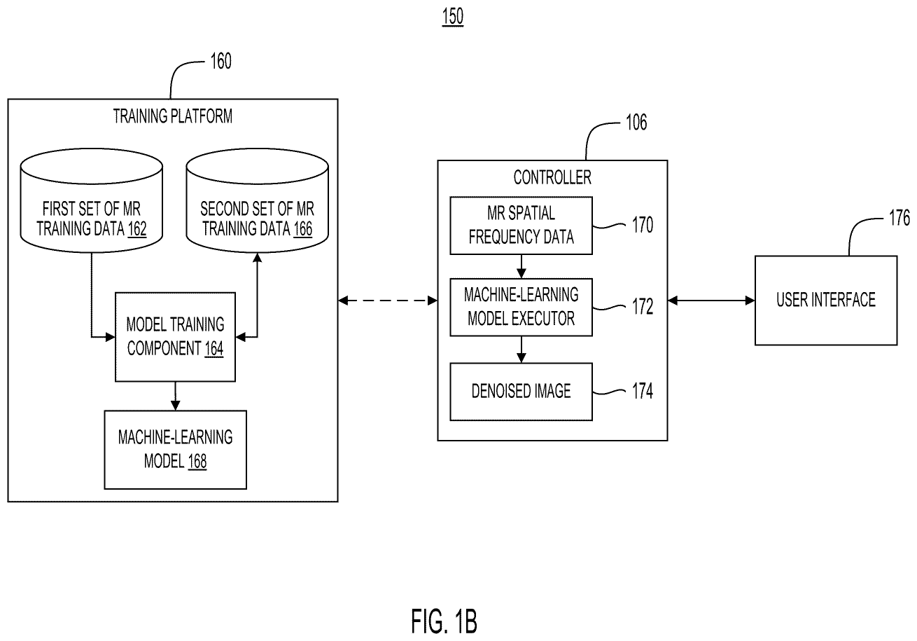

FIG. 1A illustrates an example MRI system which may be used with the denoising and dealiasing models trained using the techniques described herein. In FIG. 1A, MRI system 100 may include a computing device 104, a controller 106, a pulse sequences repository 108, a power management system 110, and magnetics components 120. The MRI system 100 is illustrative, and an MRI system may have one or more other components of any suitable type in addition to or instead of the components illustrated in FIG. 1A. Additionally, the implementation of components for a particular MRI system may differ from those described herein. Examples of low-field MRI systems may include portable MRI systems, which may have a field strength that is, in a non-limiting example, less than or equal to 0.5 T, that is less than or equal to 0.2 T, that is within a range from 1 mT to 100 mT, that is within a range from 50 mT to 0.1 T, that is within a range of 40 mT to 80 mT, that is about 64 mT, etc.

The magnetics components 120 may include B₀ magnets 122, shims 124, radio frequency (RF) transmit and receive coils 126, and gradient coils 128. The B₀ magnets 122 may be used to generate a main magnetic field B₀. B₀ magnets 122 may be any suitable type or combination of magnetics components that may generate a useful main magnetic B₀ field. In some embodiments, B₀ magnets 122 may be one or more permanent magnets, one or more electromagnets, one or more superconducting magnets, or a hybrid magnet comprising one or more permanent magnets and one or more electromagnets or one or more superconducting magnets. In some embodiments, B₀ magnets 122 may be configured to generate a B₀ magnetic field having a field strength that is less than or equal to 0.2 T or within a range from 50 mT to 0.1 T.

In some implementations, the B₀ magnets 122 may include a first and second B₀ magnet, which may each include permanent magnet blocks arranged in concentric rings about a common center. The first and second B₀ magnet may be arranged in a bi-planar configuration such that the imaging region may be located between the first and second B₀ magnets. In some embodiments, the first and second B₀ magnets may each be coupled to and supported by a ferromagnetic yoke configured to capture and direct magnetic flux from the first and second B₀ magnets.

The gradient coils 128 may be arranged to provide gradient fields and, in a non-limiting example, may be arranged to generate gradients in the B0 field in three substantially orthogonal directions (X, Y, and Z). Gradient coils 128 may be configured to encode emitted MR signals by systematically varying the B₀ field (the B₀ field generated by the B₀ magnets 122 or shims 124) to encode the spatial location of received MR signals as a function of frequency or phase. In a non-limiting example, the gradient coils 128 may be configured to vary frequency or phase as a linear function of spatial location along a particular direction, although more complex spatial encoding profiles may also be provided by using nonlinear gradient coils. In some embodiments, the gradient coils 128 may be implemented using laminate panels (e.g., printed circuit boards), in a non-limiting example.

MRI scans are performed by exciting and detecting emitted MR signals using transmit and receive coils, respectively (referred to herein as radio frequency (RF) coils). The transmit and receive coils may include separate coils for transmitting and receiving, multiple coils for transmitting or receiving, or the same coils for transmitting and receiving. Thus, a transmit/receive component may include one or more coils for transmitting, one or more coils for receiving, or one or more coils for transmitting and receiving. The transmit/receive coils may be referred to as Tx/Rx or Tx/Rx coils to generically refer to the various configurations for transmit and receive magnetics components of an MRI system. These terms are used interchangeably herein. In FIG. 1A, RF transmit and receive coils 126 may include one or more transmit coils that may be used to generate RF pulses to induce an oscillating magnetic field B₁. The transmit coil(s) may be configured to generate any type of suitable RF pulses.

The power management system 110 includes electronics to provide operating power to one or more components of the MRI system 100. In a non-limiting example, the power management system 110 may include one or more power supplies, energy storage devices, gradient power components, transmit coil components, or any other suitable power electronics needed to provide suitable operating power to energize and operate components of MRI system 100. As illustrated in FIG. 1A, the power management system 110 may include a power supply system 112, amplifier(s) 114, transmit/receive circuitry 116, and may optionally include thermal management components 118 (e.g., cryogenic cooling equipment for superconducting magnets, water cooling equipment for electromagnets).

The power supply system 112 may include electronics that provide operating power to magnetics components 120 of the MRI system 100. The electronics of the power supply system 112 may provide, in a non-limiting example, operating power to one or more gradient coils (e.g., gradient coils 128) to generate one or more gradient magnetic fields to provide spatial encoding of the MR signals. Additionally, the electronics of the power supply system 112 may provide operating power to one or more RF coils (e.g., RF transmit and receive coils 126) to generate or receive one or more RF signals from the subject. In a non-limiting example, the power supply system 112 may include a power supply configured to provide power from mains electricity to the MRI system or an energy storage device. The power supply may, in some embodiments, be an AC-to-DC power supply that converts AC power from mains electricity into DC power for use by the MRI system. The energy storage device may, in some embodiments, be any one of a battery, a capacitor, an ultracapacitor, a flywheel, or any other suitable energy storage apparatus that may bi-directionally receive (e.g., store) power from mains electricity and supply power to the MRI system. Additionally, the power supply system 112 may include additional power electronics including, but not limited to, power converters, switches, buses, drivers, and any other suitable electronics for supplying the MRI system with power.

The amplifiers(s) 114 may include one or more RF receive (Rx) pre-amplifiers that amplify MR signals detected by one or more RF receive coils (e.g., coils 126), one or more RF transmit (Tx) power components configured to provide power to one or more RF transmit coils (e.g., coils 126), one or more gradient power components configured to provide power to one or more gradient coils (e.g., gradient coils 128), and may provide power to one or more shim power components configured to provide power to one or more shims (e.g., shims 124). In some implementations, the shims 124 may be implemented using permanent magnets, electromagnetics (e.g., a coil), or combinations thereof. The transmit/receive circuitry 116 may be used to select whether RF transmit coils or RF receive coils are being operated.

As illustrated in FIG. 1A, the MRI system 100 may include the controller 106 (also referred to as a console), which may include control electronics to send instructions to and receive information from power management system 110. The controller 106 may be configured to implement one or more pulse sequences, which are used to determine the instructions sent to power management system 110 to operate the magnetics components 120 in a desired sequence (e.g., parameters for operating the RF transmit and receive coils 126, parameters for operating gradient coils 128, etc.). A pulse sequence may generally describe the order and timing in which the RF transmit and receive coils 126 and the gradient coils 128 operate to acquire resulting MR data. In a non-limiting example, a pulse sequence may indicate an order and duration of transmit pulses, gradient pulses, and acquisition times during which the receive coils acquire MR data.

A pulse sequence may be organized into a series of periods. In a non-limiting example, a pulse sequence may include a pre-programmed number of pulse repetition periods, and applying a pulse sequence may include operating the MRI system in accordance with parameters of the pulse sequence for the pre-programmed number of pulse repetition periods. In each period, the pulse sequence may include parameters for generating RF pulses (e.g., parameters identifying transmit duration, waveform, amplitude, phase, etc.), parameters for generating gradient fields (e.g., parameters identifying transmit duration, waveform, amplitude, phase, etc.), timing parameters governing when RF or gradient pulses are generated or when the receive coil(s) are configured to detect MR signals generated by the subject, among other functionality. In some embodiments, a pulse sequence may include parameters specifying one or more navigator RF pulses, as described herein.

Examples of pulse sequences include zero echo time (ZTE) pulse sequences, balance steady-state free precession (bSSFP) pulse sequences, gradient echo pulse sequences, inversion recovery pulse sequences, diffusion weighted imaging (DWI) pulse sequences, spin echo pulse sequences including conventional spin echo (CSE) pulse sequences, fast spin echo (FSE) pulse sequences, turbo spin echo (TSE) pulse sequences or any multi-spin echo pulse sequences such a diffusion weighted spin echo pulse sequences, inversion recovery spin echo pulse sequences, arterial spin labeling pulse sequences, and Overhauser imaging pulse sequences, among others.

Examples of image contrast include T1-weighted image, T2-weighted image, fluid attenuated inversion recovery (FLAIR), diffusion-weighted image (DWI) acquired at b-value of 0 s/mm² to 1000 s/mm².

As illustrated in FIG. 1A, the controller 106 may communicate with the computing device 104, which may be programmed to process received MR data. In a non-limiting example, the computing device 104 may process received MR data to generate one or more MR images using any suitable image reconstruction processes, including the execution of denoising and dealiasing machine-learning models trained using the techniques described herein. Additionally or alternatively, the controller 106 may process received MR data to generate one or more denoised and/or dealiased MR images using any suitable image denoising and/or dealiasing processes. The controller 106 may provide information about one or more pulse sequences to computing device 104 for the processing of data by the computing device. In a non-limiting example, the controller 106 may provide information about one or more pulse sequences to the computing device 104 and the computing device 104 may perform an image denoising and/or dealiasing process based, at least in part, on the provided information.

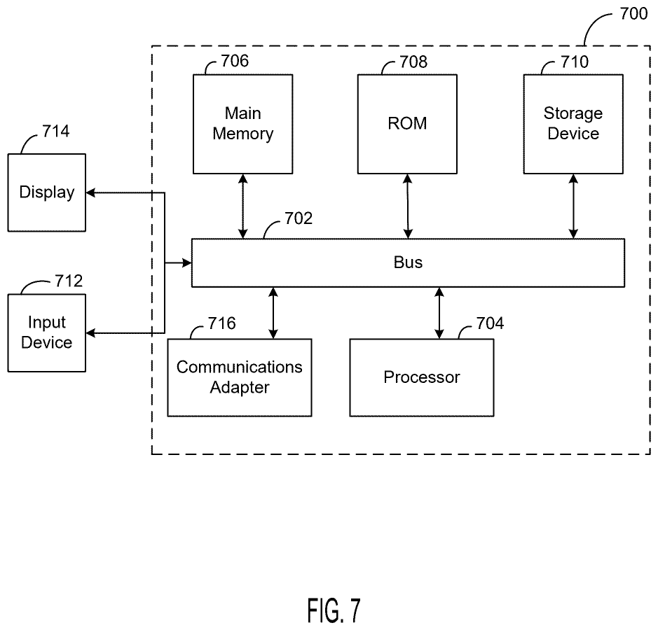

The computing device 104 may be any electronic device configured to process acquired MR data and generate one or more images of a subject being imaged. The computing device 104 may include at least one processor and a memory (e.g., a processing circuit). The memory may store processor-executable instructions that, when executed by a processor, cause the processor to perform one or more of the operations described herein. The processor may include a microprocessor, an application-specific integrated circuit (ASIC), a field-programmable gate array (FPGA), a graphics processing unit (GPU), a tensor processing unity (TPU), etc., or combinations thereof. The memory may include, but is not limited to, electronic, optical, magnetic, or any other storage or transmission device capable of providing the processor with program instructions. The memory may further include a floppy disk, CD-ROM, DVD, magnetic disk, memory chip, ASIC, FPGA, read-only memory (ROM), random-access memory (RAM), electrically erasable programmable ROM (EEPROM), erasable programmable ROM (EPROM), flash memory, optical media, or any other suitable memory from which the processor may read instructions. The instructions may include code generated from any suitable computer programming language. The computing device 104 may include any or all of the components and perform any or all of the functions of the computer system 700 described in connection with FIG. 7. In some implementations, the computing device 104 may be located in a same room as the MRI system 100 or coupled to the MRI system 100 via wired or wireless connection.

In some implementations, computing device 104 may be a fixed electronic device such as a desktop computer, a server, a rack-mounted computer, or any other suitable fixed electronic device that may be configured to process MR data and generate one or more images of the subject being imaged. Alternatively, computing device 104 may be a portable device such as a smart phone, a personal digital assistant, a laptop computer, a tablet computer, or any other portable device that may be configured to process MR data and generate one or images of the subject being imaged. In some implementations, computing device 104 may comprise multiple computing devices of any suitable type, as aspects of the disclosure provided herein are not limited in this respect. In some implementations, operations that are described as being performed by the computing device 104 may instead be performed by the controller 106, or vice-versa. In some implementations, certain operations may be performed by both the controller 106 and the computing device 104 via communications between said devices.

The MRI system 100 may include one or more external sensors 178. The one or more external sensors may assist in detecting one or more error sources (e.g., motion, noise) which degrade image quality. The controller 106 may be configured to receive information from the one or more external sensors 178. In some embodiments, the controller 106 of the MRI system 100 may be configured to control operations of the one or more external sensors 178, as well as collect information from the one or more external sensors 178. The data collected from the one or more external sensors 178 may be stored in a suitable computer memory and may be utilized to assist with various processing operations of the MRI system 100.

As described herein above, the techniques described herein may be utilized to train denoising and dealiasing machine-learning models for images in a target domain for which clean references may be unavailable. This enables the training of machine-learning models that are superior to the accuracy of supervised and unsupervised techniques in the target domain, but with the ability to perform denoising and dealiasing on images where the noise is non-i.i.d.

The training processes described herein may be utilized to train accurate models based on under-sampled and non-Cartesian MR data.

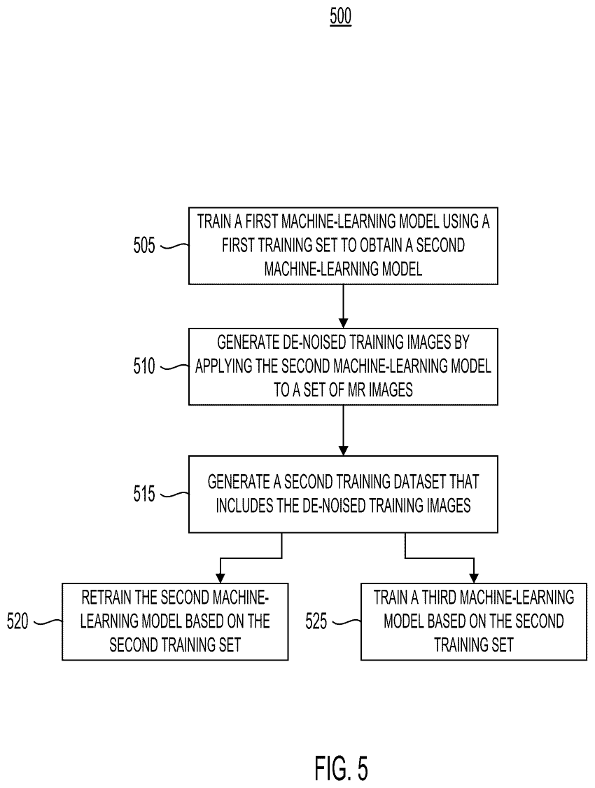

FIG. 1B illustrates an example system 150 for training a denoising and dealiasing machine-learning model to generate denoised and/or dealiased image data, in accordance with one or more implementations. In a non-limiting example, the system 150 may be used to perform all or part of the example method 500 described in connection with FIG. 5, as well as any other operations described herein. In some implementations, the system 150 forms a portion of an MRI system, such as MRI system 100 described in connection with FIG. 1A. In some implementations, the system 150 may be external to an MRI system but communicates with the MRI system (or components thereof) to perform the example method 500 of FIG. 5 as described herein.

As shown in FIG. 1B, an embodiment of the example system 150 may include the controller 106, a training platform 160, and a user interface 176. The user interface 176 may present or enable inspection of any of the reconstructed MR images generated using the techniques described herein. The user interface 176 may provide input relating to the performance such techniques, in a non-limiting example, by receiving input or configuration data relating to the training process, MR scans, or MR image reconstruction. The user interface 176 may allow a user to select a type of imaging to be performed by the MRI system (e.g., diffusion-weighted imaging, etc.), select a sampling density for the MR scan, or to define any other type of parameter relating to MR imaging or model training as described herein. In some implementations, the user interface 176 may display, via a display in communication with the user interface 176, reconstructed and denoised and/or dealiased images generated from MR data acquired by the MRI system. The user interface 176 may allow a user to initiate imaging by the MRI system, or to execute or coordinate any of the machine-learning techniques described herein.

The controller 106 may control aspects of the example system 150, in a non-limiting example, to perform at least a portion of the example method 500 described in connection with FIG. 5, as well as any other operations described herein. In some implementations, the controller 106 may control one or more operations of the MRI system, such as the MRI system 100 described in connection with FIG. 1A. Additionally or alternatively, the computing device 104 of FIG. 1A may perform some or all of the functionality of the controller 106. In such implementations, the computing device 104 may be in communication with the controller 106 to exchange information as necessary to achieve useful results.

The controller 106 may be implemented using software, hardware, or a combination thereof. The controller 106 may include at least one processor and a memory (e.g., a processing circuit). The memory may store processor-executable instructions that, when executed by a processor, cause the processor to perform one or more of the operations described herein. The processor may include a microprocessor, an ASIC, an FPGA, a GPU, a TPU, etc., or combinations thereof. The memory may include, but is not limited to, electronic, optical, magnetic, or any other storage or transmission device capable of providing the processor with program instructions. The memory may further include a floppy disk, CD-ROM, DVD, magnetic disk, memory chip, ASIC, FPGA, ROM, RAM, EEPROM, EPROM, flash memory, optical media, or any other suitable memory from which the processor may read instructions. The instructions may include code generated from any suitable computer programming language. The controller 106 may include any or all of the components and perform any or all of the functions of the computer system 700 described in connection with FIG. 7.

The controller 106 may be configured to perform one or more functions described herein. The controller 106 may store or capture MR spatial frequency data 170. The MR spatial frequency data 170 may be obtained using an MR system, such as the MRI system 100 described in connection with FIG. 1A. In some implementations, the MR spatial frequency data 170 may be obtained externally and provided to the controller 106 via one or more communication interfaces. The MR spatial frequency data 170 may be under-sampled relative to the Nyquist sampling criterion. In some embodiments, the spatial frequency domain data may include less than 90% (or less than 80%, or less than 75%, or less than 70%, or less than 65%, or less than 60%, or less than 55%, or less than 50%, or less than 40%, or less than 35%, or any percentage between 25 and 100) of the number of data samples required by the Nyquist criterion. Similarly, the MR spatial frequency data 170 may be non-Cartesian data. The MR spatial frequency data 170 may be represented in the k-space domain, as described herein. The MR spatial frequency data 170 may be generated by an MR scanner, which may utilize a suitable pulse sequence and sampling technique. In some implementations, the MR spatial frequency data 170 may be gathered using a Cartesian sampling scheme. Alternatively, MR spatial frequency data 170 may be generated using a non-Cartesian sampling scheme, such as a radial, spiral, rosette, or Lissajou sampling scheme, among others.

The controller 106 may include a machine-learning model executor 172. The machine-learning model executor 172 may execute an image reconstruction pipeline to generate reconstructed images from the MR spatial frequency data 170. The machine-learning model executor 172 may execute a denoising and dealiasing machine-learning model, such as the machine-learning model 168 (which in some implementations may be stored in memory of the controller 106 or the computing device 104) using the reconstructed images as input to generate denoised and/or dealiased images 174. The machine-learning model 168 may be similar to, or may include, any of the denoising and dealiasing models described herein. The machine-learning model 168 may be or may include a variational reconstruction network, as described herein. The machine-learning model executor 172 may execute the machine-learning model 168 using the machine-learning model 168 as input to generate a denoised and/or dealiased image 174 (e.g., as part of an image reconstruction pipeline, etc.).

The machine-learning model 168 may be trained by the training platform 160, in a non-limiting example, by implementing the example method 500 of FIG. 5. As described in further detail herein, the machine-learning model 168 may generate the denoised and/or dealiased image 174 from reconstructed images generated based on the MR spatial frequency data 170. The denoised and/or dealiased image 174 generated by the machine-learning model 168 may be presented, in a non-limiting example, for inspection by a user at the user interface 176. The denoised and/or dealiased image 174, upon generation, may be stored in one or more data structures in the memory of the controller 106.

The training platform 160 may be, or may include, the computing device 104 of FIG. 1A. Alternatively, the training platform 160 (or any components thereof) may be implemented as part of the controller 106. The training platform 160 may include at least one processor and a memory (e.g., a processing circuit). The memory may store processor-executable instructions that, when executed by a processor, cause the processor to perform one or more of the operations described herein. The processor may include a microprocessor, an ASIC, an FPGA, a GPU, a TPU, etc., or combinations thereof. The memory may include, but is not limited to, electronic, optical, magnetic, or any other storage or transmission device capable of providing the processor with program instructions. The memory may further include a floppy disk, CD-ROM, DVD, magnetic disk, memory chip, ASIC, FPGA, ROM, RAM, EEPROM, EPROM, flash memory, optical media, or any other suitable memory from which the processor may read instructions. The instructions may include code generated from any suitable computer programming language. The training platform 160 may include any or all of the components and perform any or all of the functions of the computer system 700 described in connection with FIG. 7. In some implementations, the training platform 160 may be a desktop computer, a server, a rack-mounted computer, a distributed computing environment, or any other computing system that may be configured to train the machine-learning model 168 using the de-training techniques described herein. The training platform 160 may include any number of computing devices of any suitable type.

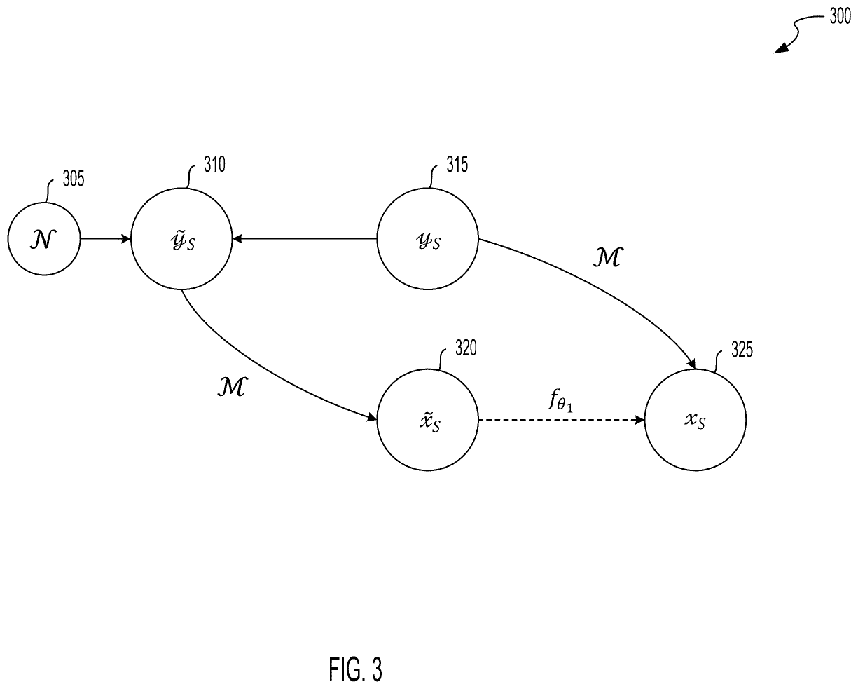

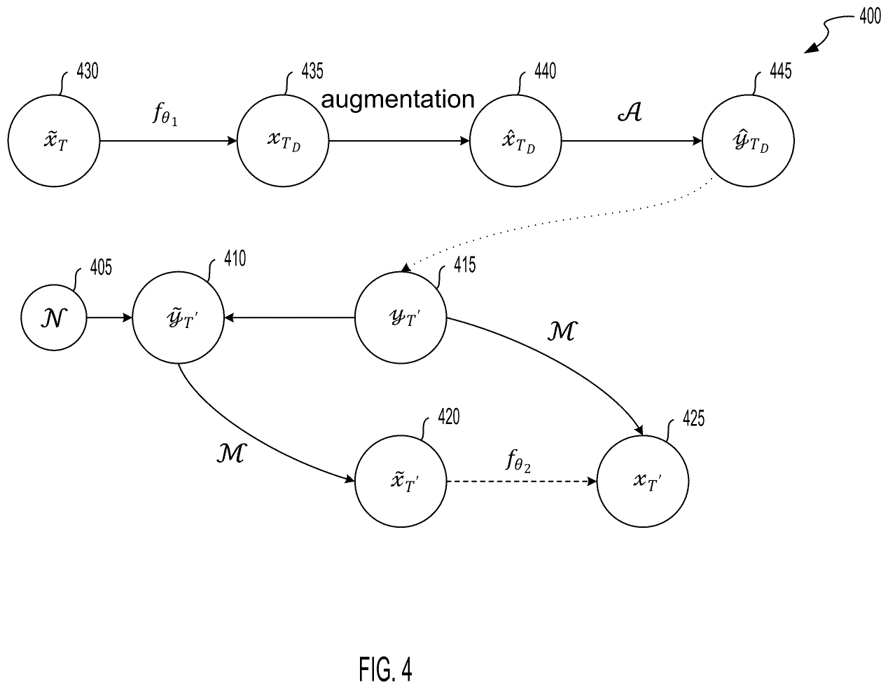

The training platform 160 may include a first set of MR training data 162, a second set of MR training data 166, a model training component 164, and the machine-learning model 168 (e.g., which may be trained and retrained as described herein by the model training component 164). The model training component 164 may be implemented using any suitable combination of software or hardware. Additionally or alternatively, the model training component 164 may be implemented by one or more servers or distributed computing systems, which may include a cloud computing system. In some implementations, the model training component 164 may be implemented using one or more virtual servers or computing systems. The model training component 164 may implement the example method 500 described in connection with FIG. 5 to train the machine-learning model 168, as well as any other operations relating to the training of the a denoising and dealiasing model as described herein. These training processes may be similar to the stages of the training process described in connection with FIGS. 3 and 4, which may be implemented by the model training component 164 to train the machine-learning model 168.

The model training component 164 may utilize the first set of MR training data 162 and the second set of MR training data 166 to train the machine-learning model 168, as described herein. The first set of MR training data 162 may store batches of MR spatial frequency data in association with respective clean reference images (e.g., images that do not include noise, or have had the noise removed). The first set of MR training data 162 may include raw data or reconstructed images that include noise that are captured using high-field MRI systems. In a non-limiting example, the first set of MR training data 162 may include images from a source domain (e.g., images captured using a different type of MR system, images captured from a particular patient population, etc.).

The first set of MR training data 162 may be previously generated by an MR scanner (e.g., include multiple historic MRI scans). The first set of MR training data 162 may include images that are reconstructed from MR spatial frequency data (e.g., k-space domain data, non-Cartesian data, etc.). The reconstructed images in the MR training data repository 162 may be augmented, in a non-limiting example, by applying affine transformations to create images with different orientation and size, by adding noise to create images with different SNR, introducing motion artifacts, incorporating phase or signal modulation for more complex sequences like echo trains, or modeling the dephasing of the data to adapt the model to a sequence-like diffusion weighted imaging.

The first set of MR training data 162 may include simulated MR images utilizing a Bloch equation and data for anatomical tissue structures. Bloch equation simulates MR pulse sequence of different parameters, including but not limited to, TR, TE, TI, bandwidth, a sequence of RF pulse excitation, gradient encoding, and generates a contrast value based on anatomical tissue parameters, such as, including but not limited to, T1, T2, T2* and Proton Density. Bloch equation generates value for each combination of tissue parameters. The final image may comprise or consist of an image with several tissues components with assigned values, hence forming a simulated image of arbitrary contrast. The contrast includes, in a non-limiting example, T1-weighted image, T2-weighted image, FLAIR image, or DWI image at different b-values (e.g. 0 s/mm²-1000 s/mm²). Anatomical tissues includes brain, skull, white matter, gray matter, lateral ventricle, amygdala, etc.

The first set of MR training data 162 may include simulated MR images utilizing MR contrast equations, such as spin-echo

$$( e . g . S = p ( 1 - {e}^{- \frac{TR}{{T}_{1}}} ) {e}^{- \frac{TE}{{T}_{2}}} ) ,$$

inversion recovery, double inversion recovery, gradient recalled echo, Stejskal-Tanner formula for diffusion weighted image, or a random number generator

The first set of MR training data 162 may include, combine, or augment with natural images acquired by camera (e.g., images of cat, images of a mountain, images of a face, etc.) to increase the diversity. In some examples, MR images may be replaced by natural images. In some examples, a subregion of MR image may be replaced by a subregion of natural images. In some examples, anatomical structure data may be combined with natural images, and only the region within some anatomical structures may be replaced by content of natural images.

The first set of MR training data 162, may include 2D images, multiple slices of 2D images, 3D images, may include MR image at different field strength (e.g., 5 mT, 64 mT, 1.5 T, 12 T), CT image, PET image, ultrasound image, simple geometric objects (circle, triangle, rectangle, trapezoid, curved lines) at different intensity scale (e.g., value 0, value 0.68, value 512). One source may be used, or two or more sources may be combined. The potential number of sources may include, for example, one, two, three, five, or 10 data sources, and the sources may be of the same kind or of different kinds. Images from the same or from different sources may have different dimensions, shapes, and/or sizes.

The model training component 164 may perform any of the functionality described herein to train the machine-learning model 168, in a non-limiting example, including performing the two-stage training process described herein. In the first training step, a supervised training process may be performed to train the machine-learning model 168, which as described herein may include a DNCNN or another suitable denoising and dealiasing model, using the first set of MR training data 162 (e.g., based on the clean reference images included therein). Once the machine-learning model 168 has been is trained, the model training component 164 may apply the machine-learning model 168 to images of a target domain (e.g., captured from a patient population corresponding to the target domain using a low-field MRI system). The images in the target domain may not necessarily be associated with clean reference images. The model training component 164 may utilize the outputs produced when executing the machine-learning model 168 over the images in the target domain as clean reference images for re-training.

The images from the target domain, including the outputs of the machine-learning model 168, may be stored as part of the second set of MR training data 166. In some embodiments, the second set of MR training data 166 may include images from the source domain (and their corresponding clean reference images) in combination with images from the target domain (e.g., noisy and corresponding clean images generated using the machine-learning model 168). In some implementations, the second set of MR training data 166 may include only images (e.g., noisy and clean) corresponding to the target domain. In some embodiments, the model training component 164 may perform data augmentation (including, e.g., image sharpening, affine transformation, elastic deformation, inserting or adding different geometric objects, and/or intensity augmentation) to increase the size of the second set of MR training data. The model training component 164 may then re-train the machine-learning model 168 using the techniques described herein based on the second set of MR training data 166. Once the machine-learning model 168 has been re-trained (e.g., the training process has terminated), the training platform 160 may provide the trained machine-learning model 170 to the controller 106, such that the machine-learning model executor 172 may use the machine-learning model 168 to generate denoised and/or dealiased images 174, as described herein.

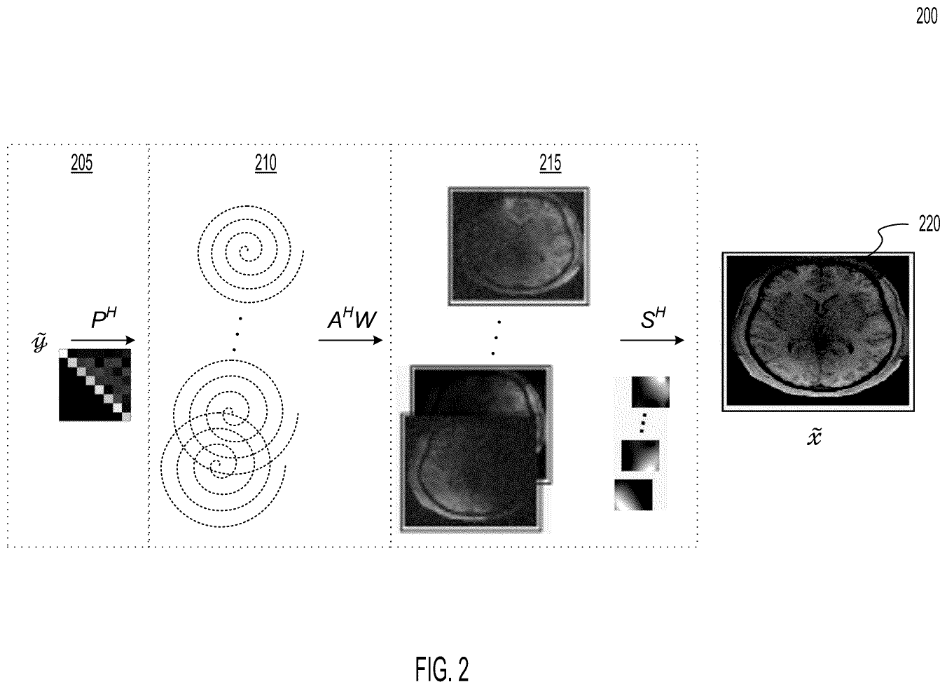

FIG. 2 depicts an example dataflow diagram 200 of an example magnetic resonance image reconstruction pipeline, in accordance with one or more implementations. The image reconstruction pipeline shown in the diagram 200 may transform raw scan data (e.g., k-space data) using mathematical operations into image data. The raw input data may be non-Cartesian scan data. The operations of the image reconstruction pipeline may be represented by the Equation 1, below.

$$\tilde{x} = ℳ ( \tilde{𝓎} ) = abs ( {S}^{H} {A}^{H} {WP}^{H} \tilde{𝓎} ) \qquad ( 1 )$$

In Equation 1 above, P^{H} corresponds to a coil de-correlation operation, A^{H}W corresponds to a gridding operation, S^{H} corresponds to a coil combination operation, and abs(·) corresponds to a magnitude operation. The image reconstruction process shown in the diagram 200 may result in spatially correlated, inhomogeneous noise in the reconstructed image due to sampling artefacts and coil correlation, as well as Rician bias. The reconstruction pipeline, as used in further operations described herein, is denoted by M.

At step 205, the raw input data [symbol] is applied to a coil de-correlation operation P^{H}. The raw input data [symbol] includes data from an MRI scan that may be converted into a visible image. The raw input data \tilde{y} includes noise (data denoted by a tilde accent herein indicates that said data includes noise). The operation P^{H} may be a transform operation, such as the Hermitian adjoint or conjugate transpose of the pre-whitening matrix P. The output of the transform operation P^{H} may be provided as input to the next stage of the image reconstruction pipeline.

At step 210, the output of the transform operation P^{H} (e.g., de-correlated medical image data) may be provided as input to the gridding operation A^{H}W. The gridding operation A^{H}W may include operations that transform the decorrelated medical image data from the spatial frequency domain (e.g., k-space data) to the image domain. The gridding operation A^{H}W may compensate for sampling density in non-Cartesian spatial frequency data. The outputs of the gridding operation A^{H}W may include one or more medical images that each correspond to a set of MR signals captured by an RF receive coil.

At step 215, the medical images generated by the gridding operation A^{H}W may be applied to the coil combination operation S^{H}. The coil combination operation S^{H} may combine the medical images, which each correspond to the MR signal responses of multiple respective RF receive coils, into a single noisy medical image designated \tilde{x}. In some embodiments, a magnitude operation may be applied to the output of the coil combination operation S^{H} to produce the noisy medical image 220 (\tilde{x}).

Once the noisy medical image 220 has been generated, the noisy medical image 220 may be provided as input to a machine-learning model (e.g., the machine-learning model 168) to generate a denoised and/or dealiased image (e.g., which may be designated as x). The machine-learning model used to generate the denoised and/or dealiased image x may be trained using the two-stage training techniques described herein. In a non-limiting example, the raw input data may correspond to a target domain for which the machine-learning model was trained using the techniques described herein.

In some embodiments, only a subset of operations of the reconstruction pipeline may be used. For example, ‘abs’ may be omitted.

In some embodiments M may be implemented by a complex MR reconstruction algorithm, such as conjugate gradient sensitivity encoding (CG-SENSE), fast iterative shrinkage-thresholding algorithm (FISTA), or alternating direction method of multipliers (ADMM).

FIG. 3 depicts an example dataflow diagram 300 of a first stage of a training process for training a denoising and dealiasing machine-learning model (e.g., the machine-learning model 168, etc.) to generate denoised and/or dealiased image data, in accordance with one or more implementations. The two-step training process described herein may be utilized when clean reference images are available on a source domain (e.g., images captured from a certain patient population or MR system) but corresponding clean reference images are unavailable on a target domain (e.g., from a clinical population using scans from low-field MR systems described herein). In the first stage of the two-stage training process, an initial model may be trained using training data available from the source domain. The trained model may subsequently be executed over noisy images from the target domain to generate denoised and/or dealiased images, which may be used as part of a second set of training data for the subsequent training step. Data augmentation, such as image sharpening, may be performed to inflate the second set of training data. Non-limiting examples of data augmentation techniques include affine transformation, elastic deformation, inserting or adding different geometric objects, and/or intensity augmentation, among others. The initial model may then be retained based on the denoised and/or dealiased images generated from the noisy target domain data, rather than using simulated image corruption (e.g., noise data, acquiring MR frequency data at sub-Nyquist rate to simulate aliasing artifacts, etc.). This improves the accuracy of the denoising and/or dealiasing process, since the second step of the training is using data from the target domain as used in inference and testing, rather than using simulated data in training and the target domain data in inference and testing.

As shown, a first training set 310 of noisy images [symbol] from a source domain is generated by adding simulated structured noise 305 to the clean reference data 315 ( [symbol] ) from the source domain. The structured noise 305 may be simulated, in a non-limiting example, by generating Gaussian noise and adding the Gaussian noise to the clean reference data 315. Other noise may also be generated, such as noise from a Poisson distribution, among other types of random structured noise. The clean reference data 315 may be non-Cartesian frequency-domain data (e.g., k-space data) from previous MR scans. Noisy images 320 ( [symbol] ) are generated by propagating the first training set through an image reconstruction pipeline, [symbol] (e.g., the reconstruction pipeline described in connection with FIG. 2, etc.). Corresponding clean reference images 325 (x_{S}) may be generated using similar techniques (e.g., propagating the clean reference data 315 through the reconstruction pipeline [symbol] ).

Using the generated noisy images 320 and the clean reference images 325 from the source domain, the initial machine-learning model f_{θ}_{₁} may be trained using a suitable training technique. The initial machine-learning model f_{θ}_{₁} may be, in a non-limiting example, the machine-learning model 168 described in connection with FIG. 1B. In one embodiment, the initial machine-learning model f_{θ}_{₁} may be a DNCNN with a number of convolutional layers (e.g., one layer, five layers, twenty layers, 100 layers). Each convolutional layer may have a predetermined kernel size (e.g., 3 by 3, 5 by 1, 14 by 15, 4 by 5 by 6) and a predetermined stride or bias term. In some implementations, each convolution layer may apply 64 filters (or, e.g., 32 filters, 128 filters, 256 filters, 383 filters, 1024 filters, 5321 filters) to the data produced by the preceding layer. In some implementations, the machine-learning model f_{θ}_{₁} may include one or more activation layers (e.g., a rectified linear unit (ReLU), leaky ReLU, parametric ReLU, sigmoid, tanh, exponential linear unit (ELU), generalized ELU (GELU) softmax etc.) disposed between the convolutional layers. In some implementations, the machine-learning model may include one or more skip connections. The initial machine-learning model f_{θ}_{₁} may include an input layer that receives a noisy image as input, and produces a denoised and/or dealiased image at an output layer. Any suitable number of convolutional layers, activation layers, or other types of neural network layers (e.g., pooling layers, global average pooling layers, fully connected layers, attention layers, etc.) may be included in the initial machine-learning model f_{θ}_{₁}.

In some embodiments, the initial machine-learning model f_{θ}_{₁} may contain one or more pooling layers (e.g. 2×2 average pooling, 3×1 max pooling, 15×5 median pooling) to downsample an image to aggregate local spatial information for the network to learn context of a larger receptive field. The initial machine-learning model f_{θ}_{₁} may contain unpooling layers (e.g. 2×2 average unpooling,) or interpolation layer (linear interpolation, bilinear interpolation, trilinear interpolation, spline interpolation, etc.) to upsample the intermediate network output to generate the final output that is the same size as the input. The initial machine-learning model f_{θ}_{₁} may include normalization layers (e.g. batch-norm, instance-norm, group-norm layers, etc.).

In some embodiments, the initial machine-learning model f_{θ}_{₁} may contain one or more discrete-wavelet-transform (DWT) layers for pooling and inverse-discrete-wavelet-transform (IDWT) layers for unpooling. A DWT layer generalizes average pooling layer by instead of collapsing the local patch information (e.g. patch size 2×2 pixels, or 3×3 pixels, or 4×5×6 pixels) into an average value, it outputs 4 values for 2D patch: For the case of Haar wavelet, these 4 are: average value, vertical image gradient, horizontal image gradient, and diagonal image gradient. IDWT unpooling takes 4 values, typically represented as network channels, and combine them into one by applying IDWT. DWT and IDWT layers allow the machine learning model to retain high-frequency information at coarser layers of the network, hence improving the model performance. DWT and IDWT may use such wavelets as Haar, Daubechies 4, Daubechies 10, biorthogonal 1.3 biorthogonal 2.2, morlet, etc. for the basis to generate the 4 values. For general wavelet basis, one-dimensional wavelet transform low-frequency band (L) and high frequency band (H) for the features. For two dimensions, four features are Low frequency band in two directions (LL), low frequency band in one and high frequency band in one direction (HL, LH), and high frequency bands for both dimensions (HH). In some embodiments, the DWT layers generate 8 values for 3D patch: LLL, LLH, LHL, LHH, HLL, HLH, HHL, HHH. Each wavelet basis generates different values for each of the low and high band.

In some embodiments, the initial machine-learning model f_{θ}_{₁} may process two-dimensional (2D) image (e.g. 32×32, 100×100, 256×512 pixels), multi 2D-slice (2.5D) image (e.g. 100×100×3 pixels, 32×40×5 pixels, 512×512×13 pixels), or 3D volume (e.g. 100×100×100 pixels or 32×33×34 pixels). 2D images may be processed by a machine-learning model comprised of one or more 2D convolutional layers, 2D nonlinearity layers, 2D normalization layers, 2D pooling layers and 2D DWT/IDWT layers. 2.5D images may be processed by a machine-learning comprised of one or more 2D convolutional layers, 2D nonlinearity layers, 2D normalization layers, 2D pooling layers and 2D DWT/IDWT layers, where multiple slices (e.g. 3 slices, 5 slices, 7 slices, 11 slices, 32 slices, 100 slices) are represented as input channels (much like color image is represented as 3 channel input). 3D image may be processed by a machine-learning model comprised of one or more 3D convolutional layers, 3D nonlinearity layers, 3D normalization layers, 3D pooling layers and 3D DWT/IDWT layers.

In one embodiment, the initial machine-learning model f_{θ}_{₁} may be 2.5D multi-level wavelet CNN, which takes 2.5D image as an input, and processes the image through encoding and decoding pathways. Encoding pathways may comprise of one or more convolutional layers, nonlinearity layers, normalization layers, DWT layers with, for example, 1 to 10 downsampling levels. Decoding pathway takes the downsampled feature representations at each level and applies one or more of IDWT layers, convolution layers, nonlinearity layers, and normalization layers.

In some embodiments, the initial machine-learning model may only learn to perform denoising. In some embodiments, the initial machine-learning model may learn to perform denoising and dealiasing. In one embodiment, the initial machine-learning model may only learn to perform dealiasing. In one embodiment, the initial machine-learning model may only learn to perform sharpening when aliasing represents signal blurrying. In one embodiment, the initial machine-learning model may only learn to perform upsampling when aliasing represents signal with reduced high frequency content and hence reduced resolution. In one embodiment, the initial machine-learning model may only learn to perform motion correction when aliasing represents the data shifted by multiplying complex exponentials.

Training the initial machine-learning model f_{θ}_{₁} may include performing a supervised learning process (e.g., stochastic gradient descent and backpropagation, an Adam optimizer, etc.) to iteratively adjust the trainable parameters of the initial machine-learning model f_{θ}_{₁}. Any suitable loss function may be utilized to train the initial machine-learning model f_{θ}_{₁}, such as an L1 loss, an L2 loss, a mean-squared error (MSE) loss, binary cross entropy (BCE), categorical cross entropy (CC), or sparse categorical cross entropy (SCC) loss functions, among others. The loss may be calculated based on the output of the machine-learning model f_{θ}_{₁} when a noisy image 320 is provided as input and based on a corresponding clean reference image 325. The supervised training process is illustrated by the dotted arrow in the diagram 300, indicating that the machine-learning model f_{θ}_{₁} is trained to generate the corresponding clean reference images 325 from input noisy images 320 (which are generated by adding structured noise as described herein). Once the machine-learning model f_{θ}_{₁} has been trained, the machine-learning model f_{θ}_{₁} may be utilized in the second stage of the training process described in connection with FIG. 4.

FIG. 4 depicts an example dataflow diagram 400 of a second stage of the training process for re-training a denoising and dealiasing machine-learning model (e.g., the machine-learning model 168, the machine-learning model f_{θ}_{₁} described in connection with FIG. 3, etc.) to generate denoised and/or dealiased image data, in accordance with one or more implementations. FIG. 4 shows the second stage in the two-stage training process, in which the machine-learning model f_{θ}_{₁} is used to generate clean images in a target domain (e.g., clinical data captured using low-field MRI systems) from noisy images in the target domain. The clean images are used with the corresponding noisy images in the clinical domain to re-train the machine-learning model f_{θ}_{₁}, thereby obtaining a re-trained machine-learning f_{θ}_{₂}.

As shown in the diagram 400, noisy images (e.g., captured using a low-field MR system) from the target domain 430 (designated \tilde{x}_{T}) are provided as input to the machine-learning model f_{θ}_{₁}, which is executed to produce the clean images from the target domain 435 (designated x_{T}). Executing the machine-learning model f_{θ}_{₁} may include propagating the input data (e.g., each noisy image from the target domain 430) through the machine-learning model f_{θ}_{₁}. At this stage in the training process, the machine-learning model f_{θ}_{₁} has been trained on images from the source domain, as described in connection with FIG. 3. Each noisy image from the target domain 430 may be provided to the machine-learning model f_{θ}_{₁} to produce a corresponding set of clean images from the target domain 435.

In some embodiments, the target domain may include images from fast spin echo (FSE) diffusion-weighted imaging (DWI) sequence acquired at low-field strength (e.g. 64 mT, 1 mT to 700 mT) at b-value of, but not restricted to, 0 s/mm² and 900 s/mm².

In some embodiments, the target domain may include T1-weighted image, T2-weighted image, and/or FLAIR.

To produce a second set of training data (e.g., the second set of MR training data 166 of FIG. 1B, etc.) to retrain the machine-learning model f_{θ}_{₁}, data augmentation may be performed to increase the size of the clean images generated for the target domain 435. A non-exhaustive list of example of data augmentation processes that may be applied to the images generated by the machine-learning model f_{θ}_{₁} include image sharpening (e.g., using random Gaussian kernels), various transformations (e.g., rotation, cropping, horizontal or vertical flipping), or other data augmentation techniques (e.g., affine transformation, elastic deformation, inserting or adding different geometric objects, intensity augmentation, etc.). One or more of the data augmentation techniques may also be performed on the input noisy images from the target domain, where appropriate. Data augmentation may be used to both remove some remaining noise (e.g., image sharpening) or to increase the size of the training dataset (e.g., duplication and horizontal/vertical flipping). Performing the data augmentation techniques on the clean images of the target domain 435 produces the augmented clean images of the target domain 440 (designated \hat{x}_{T}_{_{D}}).

Once the augmented clean images of the target domain 440 have been generated using the data augmentation techniques, the transformation [symbol] may be performed on each augmented clean image of the target domain 440 to generate corresponding augmented clean frequency data of the target domain 445 (designated [symbol] ). As described herein, the augmented clean frequency data of the target domain 445, along with the noisy images from the target domain 430, may be utilized to generate a second training set to retrain the machine-learning model f_{θ}_{₁}.

The corresponding set of clean images may be utilized as part of the second training set 415 (designated as [symbol] ) to retrain the machine-learning model f_{θ}_{₁}. The second training set 415 may include spatial frequency data from the target domain (e.g., the augmented clean frequency data of the target domain 445), as well as the spatial frequency data corresponding to clean reference images from the source domain (e.g., the clean reference data 315). As such, the second training set 415 may include spatial frequency data from the source domain as well as clean spatial frequency data generated for the target domain. In some embodiments, the second training set 415 includes only the clean spatial frequency data corresponding to the target domain (e.g., the augmented clean frequency data of the target domain 445). In some embodiments, using techniques similar to those described herein, simulated noise 405 may be added to the second training set 415. In one embodiment, simulated noise 405 is added to the second training set 415 to generate the second set of noisy spatial frequency data 410 (designated as [symbol] ). In some other embodiments, noise is not added to the second training set 415, and a transform (e.g., from the image to the frequency domain) of the noisy images of the target domain 430 are utilized as the second set of noisy spatial frequency data 410. The simulated noise 405 may be any type of simulated image corruption, including simulated noise or MR frequency data that is acquired at sub-Nyquist rates to simulate aliasing artifacts.

The simulated noise 405 may be any type of simulated image corruption, including simulated external interferences (zipper line), adding or multiplying a subset of frequency data by an offset or a different scale. The simulated noise 405 may be reduced image resolution by attenuating the high frequency information. The simulated noise 405 may be ringing by multiplying the frequency data by an indicator function that is square-shaped. The simulated noise 405 may be patient motion obtained by multiplying the frequency data by complex exponentials.

Corresponding reconstruction transforms [symbol] may be applied to the second set of noisy spatial frequency data 410 and the second training set 415 to generate the second set of noisy images 420 (designated \tilde{x}_{T′}) and the second set of clean images 425, respectively. In embodiments where the transform (e.g., from the image to the frequency domain) of the noisy images of the target domain 430 are utilized as the second set of noisy spatial frequency data 410, the noisy images from the target domain 430 and the augmented clean images of the target domain 440 may be utilized as the second set of noisy images 420 and the second set of clean images 425, respectively, to re-train the machine-learning model f_{θ}_{₁}.

In some embodiments, a machine-learning model may be trained to only perform denoising of non i.i.d noise. In some embodiments, a machine-learning model may be trained to only perform dealiasing.

In some embodiments, a denoising and aliasing network may process both noisy data [symbol] and \tilde{x}_{T′} and use [symbol] and x_{T′} as the second set of clean images.

In some embodiments, a dealiasing machine-learning model may be trained by generating pairs of [symbol] and \tilde{x}_{T′} as the noisy input and use [symbol] and x_{T′} as the clean image. The second set of noisy data may be generated by removing a subset of MR frequency data (e.g. 50%) [symbol] to simulate aliasing artifact hence creating an aliased image \tilde{x}_{T′} by applying reconstruction pipeline [symbol] to [symbol] . The alias-free data x_{T′} may be generated by applying reconstruction pipeline [symbol] to [symbol] .

In some embodiments, one or more functions in [symbol] may be part of the machine-learning model. In some embodiments, one or more functions in [symbol] may be replaced by trainable parameters (e.g. convolutional layers, fully-connected layers, learnable parameters).

In some embodiments, a machine-learning model may include pluralities of data consistency layers and denoising networks (e.g. DNCNN).

In some embodiments, a machine learning model may a model-based deep learning model (MoDL), which utilizes an advanced algorithm for the data consistency layer. In some embodiments, the advanced algorithm may form an optimization algorithm to minimize data consistency, which is solved by conjugate gradient (CG) descent algorithm. In some embodiments, the data consistency layer may include sample density compensation W and spectral normalization layer.

Once the second set of noisy images 420 and the second set of clean images 425 have been generated, the machine-learning model machine-learning model f_{θ}_{₁} may be-retrained to obtain the re-trained machine-learning model f_{θ}_{₂}. In one embodiment, re-training the machine-learning model f_{θ}_{₁} may include re-training the machine-learning model f_{θ}_{₁} from scratch (e.g., starting from default values for the trainable parameters for the machine-learning model f_{θ}_{₁}. In some embodiments, re-training the machine-learning model f_{θ}_{₁} may include training the machine-learning model f_{θ}_{₁} using the trainable parameters of the machine-learning model f_{θ}_{₁} following the first stage of the training process described in FIG. 3.

Retraining the machine-learning model f_{θ}_{₁} may include performing a supervised learning process (e.g., stochastic gradient descent and backpropagation, an Adam optimizer, etc.) to iteratively adjust the trainable parameters of the machine-learning model f_{θ}_{₁}, eventually obtaining the re-trained machine-learning model f_{θ}_{₂}. As described herein, any suitable loss function may be utilized to re-train the machine-learning model f_{θ}_{₁}, such as an L1 loss, an L2 loss, an MSE loss, BCE loss, a CC loss, or a SCC loss, among others. The loss may be calculated based on the output of the machine-learning model f_{θ}_{₁} when a noisy image from the second set of noisy images 420 is provided as input and based on a corresponding clean reference image from the second set of clean images 425. The supervised training process is illustrated by the dotted arrow in the diagram 400, indicating that the re-trained machine-learning model f_{θ}_{₂} is trained to generate the corresponding second set of clean images 425 from the second set of noisy images 420.

FIG. 5 illustrates a flowchart of an example method 500 of training a machine-learning model (e.g., the machine-learning model 168 of FIG. 1B, etc.) to generate denoised and/or dealiased MR images using a two-stage supervised learning process, in accordance with one or more implementations. The method 500 may be executed using any suitable computing system (e.g., the training platform 160, the controller 106, or the computing device 104 of FIG. 1, the computing system 700 of FIG. 7, etc.). It will be appreciated that certain steps of the method 500 may be executed in parallel (e.g., concurrently) or sequentially, while still achieving useful results. The method 500 may be executed iteratively to update or otherwise train the denoising and dealiasing machine-learning model, as described herein.

The method 500 may include act 505, in which a first machine-learning model (e.g., the machine-learning model 168) is initially trained using a first training set (e.g., the first set of MR training data 162) corresponding to a source domain to obtain a second machine-learning model. As described herein, the source domain may include images captured from a patient population that is different from images corresponding to a target domain. The source domain may include images captured using a different type of MR system than the target domain or images captured from a particular patient population. The images corresponding to the source domain may be generated, in a non-limiting example, by applying raw MR scan data (e.g., spatial frequency data) to an image reconstruction pipeline, such as the image reconstruction pipeline described in connection with FIG. 2. Simulated noise data may be added to the raw scan data prior to reconstruction in order to simulate noisy images, which may be paired with a corresponding clean image (without simulated noise) to serve as a reference for supervised learning. The simulated noise data may be any type of simulated image corruption. In an embodiment, the images in the first training set may be augmented, in a non-limiting example, by applying affine transformations to create images with different orientation and size, by adding noise to create images with different SNR, introducing motion artifacts, incorporating phase or signal modulation for more complex sequences like echo trains, or modeling the dephasing of the data to adapt the model to a sequence-like diffusion weighted imaging.

The first and second machine-learning models may be DNCNN models with a number of convolutional layers (e.g., twenty convolutional layers). Each convolutional layer may have a predetermined kernel size (e.g., 3 by 3) and a predetermined stride or bias term. In some implementations, each convolution layer may apply 64 filters to the data produced by the preceding layer. Training the first machine-learning model may include performing a supervised learning process (e.g., stochastic gradient descent and backpropagation, an Adam optimizer, etc.) to iteratively adjust the trainable parameters of the first machine-learning model, in a non-limiting example, until a predetermined training termination condition has been reached (e.g., predetermined model accuracy has been achieved, a predetermined amount of training data has been used to train the model, etc.). Any suitable loss function may be utilized to train the first machine-learning model, such as an L1 loss, an L2 loss, an MSE loss, a BCE loss, a CC loss, or a SCC loss function, among others. The loss may be calculated based on the output of the first machine-learning model when a noisy image from the first training set is provided as input compared to the corresponding clean reference image in the first training set. The first machine-learning model, once trained using the first training set, is referred to as the second machine-learning model.

The method 500 may include act 510, in which denoised and/or dealiased training images corresponding to a target domain (e.g., the clean images of the target domain 435) are generated using the second machine-learning model obtained in act 505. The denoised and/or dealiased training images may be generated based on noisy images captured using a low-field MR system or a point-of-care (POC) MR imaging system. The noisy images corresponding to the target domain may include non-independent and non-identically distributed noise. To generate the clean target domain images, the second machine-learning model obtained in act 505 may be executed to produce using the noisy images corresponding to the target domain. Executing the second machine-learning model may include propagating each noisy image from the target domain through the trained second machine-learning model until a corresponding clean output image is produced.

The method 500 may include act 515, in which a second training dataset is generated. The second training set may include the denoised and/or dealiased training images generated in act 515, or images derived therefrom. The second training dataset may be generated, in a non-limiting example, by performing a data augmentation process on the images from the target domain (e.g., noisy and/or clean images, as appropriate). Data augmentation may be used to both remove some remaining noise (e.g., image sharpening) or to increase the size of the training dataset (e.g., duplication and horizontal/vertical flipping). A non-exhaustive list of example of data augmentation processes that may be applied to the images corresponding to the target domain include image sharpening (e.g., using random Gaussian kernels), various transformations (e.g., rotation, cropping, horizontal or vertical flipping), or other data augmentation techniques. The clean images corresponding to the target domain, along with their augmented variants, may be included as part of the second training dataset, which is used in act 520 to re-train the second machine-learning model, or in act 525 or to train a third machine-learning model.

The method 500 may include act 520, in which the second machine-learning model trained in act 505 is retrained based on the second training set. In some implementations, simulated noise data may be added to the clean images corresponding to the target domain in order to train re-train the second machine-learning model. The simulated noise data may be any type of simulated image corruption. In a non-limiting example, the clean images with simulated noise may be propagated through the second machine-learning model, which is then trained based on a loss calculated using the corresponding clean image of the target domain. In another embodiment, the corresponding noisy images of the target domain from which the clean images were generated may be utilized as input data that is propagated through the second machine-learning model, which is then re-trained using a calculated loss function as described herein.

The second machine-learning model may be re-trained from scratch, or may be re-trained according to its state after being trained on the source domain. In some implementations, the second machine-learning model may be trained based on the second training set using an overfitting process. Retraining the second machine-learning model may include performing a supervised learning process (e.g., stochastic gradient descent and backpropagation, an Adam optimizer, etc.) to iteratively re-adjust the trainable parameters of the second machine-learning model, eventually obtaining a re-trained machine-learning model. The re-trained machine-learning model may then be deployed and executed using patient images captured using low-field MR imaging systems or POC MR imaging systems, to obtain denoised and/or dealiased patient images.

The method 500 may include act 525, in which a third machine-learning model is trained in act 505 is retrained based on the second training set. In some implementations, the third machine-learning model may have the same architecture as the second machine-learning model. In an alternative embodiment, the third machine-learning model may have a different architecture from the second machine-learning model. The third machine-learning model may be trained based on the second training set. In some implementations, simulated noise data may be added to the clean images corresponding to the target domain in order to train the third machine-learning model. The simulated noise data may be any type of simulated image corruption. In a non-limiting example, the clean images with simulated noise may be propagated through the third machine-learning model, which is then trained based on a loss calculated using the corresponding clean image of the target domain. In another embodiment, the corresponding noisy images of the target domain from which the clean images were generated may be utilized as input data that is propagated through the third machine-learning model, which is then trained using a calculated loss function as described herein. Training the third machine-learning model may include performing a supervised learning process (e.g., stochastic gradient descent and backpropagation, an Adam optimizer, etc.) to iteratively re-adjust the trainable parameters of the machine-learning model, eventually obtaining a trained, fourth machine-learning model. The third machine-learning model may be deployed and executed using patient images captured using low-field MR imaging systems or POC MR imaging systems, to obtain denoised and/or dealiased patient images.

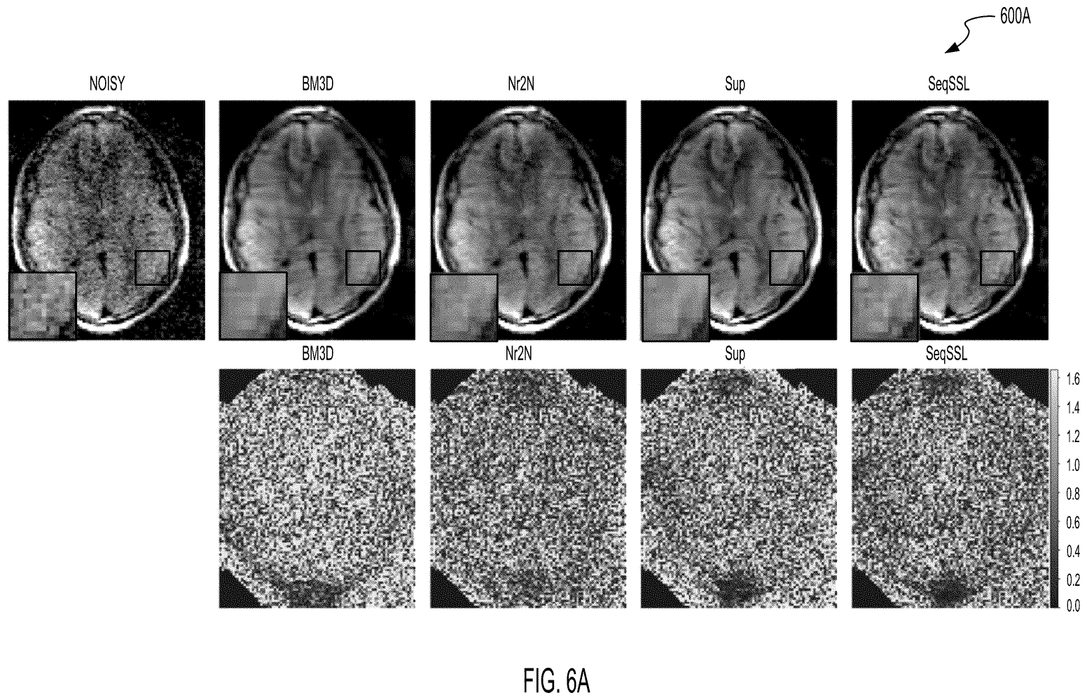

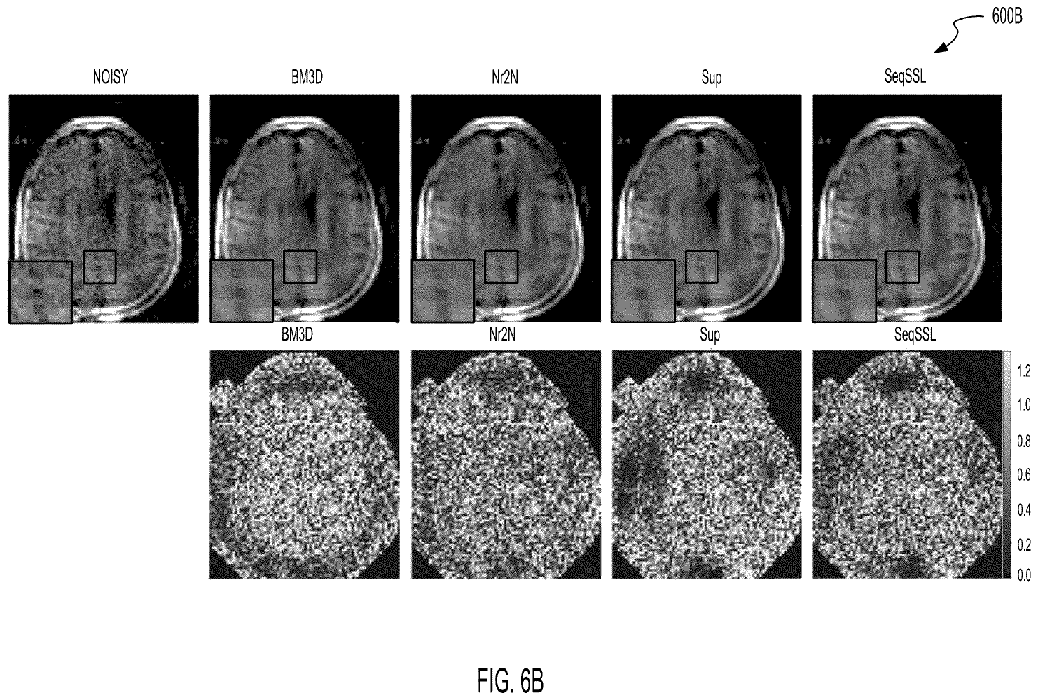

FIGS. 6A and 6B show example experimental data comparing approaches to denoise and/or dealias magnetic resonance images, in accordance with one or more implementations. FIG. 6A shows an example comparison of outputs from different denoising and/or dealiasing implementations. In FIGS. 6A and 6B, depicted is a respective noisy MR image generated from MR data acquired using a DWI pulse sequence and four corresponding denoised and/or dealiased MR images generated from the noisy MR image using various denoising and/or dealiasing approaches. Each MR image shows a zoomed-in portion of the corresponding MR image (each larger box is a zoom into each corresponding smaller box). The denoising and/or dealiasing approaches include Block-Matching and 3D filtering (BM3D), Nr2N, supervised learning (Sup) using images from a source domain (e.g., the machine-learning model trained after the first stage in FIG. 3), and sequential semi-supervised learning (SeqSSL), which is the re-trained machine-learning model generated using the two-stage training process described herein. For BM3D, hyper-parameters were manually tuned for each slice. For training the Nr2N model, the model was trained to predict images at σ=0.05 from an input with noise level of σ=0.1.

The SeqSSL model was trained using 400 cases of T1-weighted and T2-weight images from the Human Connectome Project (HCP), along with 400 cases of T1-weighted, T2-weighted, and fluid attenuated inversion recovery (FLAIR) images acquired at 64 mT, as the image data from the source domain. The image data from the target domain included DWI images captured using a low-field (e.g., 64 mT) MR system. In particular, the target domain included 289 and 308 DWI images at b=890 s/mm² and b=0 s/mm², with TE/TR=24 ms/34 ms, and a resolution of 2×2×6 mm³. The scan time was 8 minutes for b=890 s/mm² and 1.5 minutes for b=0 s/mm². The architecture of the machine-learning model used was a bias-free DNCNN with 20 convolutional layers. Patch-based training was utilized with an L1 and a structural similarity index (SSIM), using an Adam optimizer.

As shown in FIGS. 6A and 6B, the results from BM3D results were competitive with the present techniques; however, the BM3D approach required careful, manual parameter tuning. Nr2N resulted in inconsistent levels of denoising and/or dealiasing. This may be due to the fact that in practice, the noise variance present in the images is variable, whereas Nr2N requires training at a fixed noise level. Some degree of over-smoothing may be observed in Sup. The SeqSSL alleviated the issue of over-smoothing and behaved more consistently across all images.

The proposed error-correcting and/or artifact-correcting framework was qualitatively evaluated by four expert graders with backgrounds in either MR physics, clinical science, and/or radiology. Images before and after denoising and/or dealiasing were shown to the raters as a side-by-side comparison. The users were asked to rate if the denoised and/or dealiased image was “Far better”, “Clearly Better”, “Same”, “Clearly Worse”, or “Far Worse”, in terms of noise, sharpness and overall quality. The raters were also asked if the denoised and/or dealiased image had consistent image features as the input in terms of contrast, geometric fidelity, and whether artifacts were introduced. The results from this qualitative evaluation are provided below in Tables 1 and 2.

TABLE 1

| | | Clearly | | Clearly | |
|---|---|---|---|---|---|
| DWI | Far worse | worse | Same | better | Far better |
| Noise | 0 | 0 | 9 | 53 | 18 |
| Sharpness | 0 | 0 | 46 | 21 | 13 |
| Overall | 0 | 0 | 14 | 50 | 16 |

TABLE 2

| | Yes | No |
|---|---|---|
| Consistent Contrast | 80 | 0 |
| Consistent Geometric Fidelity | 80 | 0 |
| No Artefacts | 80 | 0 |

As shown in the above-results, all of the \ graders indicated “Same”, “Clearly Better”, “Far Better” for all categories. At least 88.8%, 42.5%, 82.5% voted “Clearly Better”, “Far better” for reduced noise, sharpness and overall quality, respectively. The raters also scored “Yes” for all consistency questions.

FIG. 7 is a component diagram of an example computing system suitable for use in the various implementations described herein, according to an example implementation. In a non-limiting example, the computing system 700 may implement a computing device 104, the controller 106, or the training platform 160 of FIGS. 1A and 1B, or various other example systems and devices described in the present disclosure.

The computing system 700 includes a bus 702 or other communication component for communicating information and a processor 704 coupled to the bus 702 for processing information. The computing system 700 also includes main memory 706, such as a RAM or other dynamic storage device, coupled to the bus 702 for storing information, and instructions to be executed by the processor 704. Main memory 706 may also be used for storing position information, temporary variables, or other intermediate information during execution of instructions by the processor 704. The computing system 700 may further include a ROM 708 or other static storage device coupled to the bus 702 for storing static information and instructions for the processor 704. A storage device 710, such as a solid-state device, magnetic disk, or optical disk, is coupled to the bus 702 for persistently storing information and instructions.

The computing system 700 may be coupled via the bus 702 to a display 714, such as a liquid crystal display, or active matrix display, for displaying information to a user. An input device 712, such as a keyboard including alphanumeric and other keys, may be coupled to the bus 702 for communicating information, and command selections to the processor 704. In another implementation, the input device 712 has a touch screen display. The input device 712 may include any type of biometric sensor, or a cursor control, such as a mouse, a trackball, or cursor direction keys, for communicating direction information and command selections to the processor 704 and for controlling cursor movement on the display 714.

In some implementations, the computing system 700 may include a communications adapter 716, such as a networking adapter. Communications adapter 716 may be coupled to bus 702 and may be configured to enable communications with a computing or communications network or other computing systems. In various illustrative implementations, any type of networking configuration may be achieved using communications adapter 716, such as wired (e.g., via Ethernet), wireless (e.g., via Wi-Fi, Bluetooth), satellite (e.g., via GPS) pre-configured, ad-hoc, LAN, WAN, and the like.

According to various implementations, the processes of the illustrative implementations that are described herein may be achieved by the computing system 700 in response to the processor 704 executing an implementation of instructions contained in main memory 706. Such instructions may be read into main memory 706 from another computer-readable medium, such as the storage device 710. Execution of the implementation of instructions contained in main memory 706 causes the computing system 700 to perform the illustrative processes described herein. One or more processors in a multi-processing implementation may also be employed to execute the instructions contained in main memory 706. In alternative implementations, hard-wired circuitry may be used in place of or in combination with software instructions to implement illustrative implementations. Thus, implementations are not limited to any specific combination of hardware circuitry and software.

Various example embodiments include, without limitation:
Embodiment AA: A method comprising training a denoising and dealiasing machine-learning (ML) model to generate denoised and/or dealiased imaging data, wherein training the denoising and dealiasing ML model comprises: (1) training a first ML model using a first training dataset comprising first image data to obtain a second ML model; and (2) training (a) the second ML model or (b) a third ML model using a second training dataset to obtain a fourth ML model, wherein the second training dataset comprises (i) the first image data and (ii) training image data obtained by applying at least one of the second ML model or the third ML model to second image data, and wherein the denoising and dealiasing ML model is either the fourth ML model or derived from the fourth ML model.
Embodiment AB: The method of Embodiment AA, wherein at least one of step (1) or step (2) comprises a supervised training process.
Embodiment AC: The method of either Embodiment AA or AB, wherein in step (2) the fourth ML model is obtained by using the second training dataset to train the third ML model, and wherein the third ML model has an architecture that differs from that of the second ML model.
Embodiment AD: The method of any of Embodiments AA to AC, wherein the second image data comprises noise.
Embodiment AE: The method of Embodiment AD, wherein the noise comprises non-independent and non-identically distributed noise.
Embodiment AF: The method of any of Embodiments AA to AE, further comprising applying the denoising and dealiasing ML model to a patient image to obtain a denoised and/or dealiased patient image.
Embodiment AG: The method of Embodiment AF, wherein the patient image is acquired using at least one of a low-field magnetic resonance (MR) imaging system or a point-of-care (POC) MR imaging system.
Embodiment AH: The method of any of Embodiments AA to AG, wherein the first image data and the second image data belong to separate domains.
Embodiment AI: The method of any of Embodiments AA to AH, further comprising augmenting the training image data based on an augmentation process before step (2).
Embodiment AJ: The method of any of Embodiments AA to AI, wherein the augmentation process comprises any combination of image sharpening, affine transformation, elastic deformation, inserting or adding different geometric objects, or intensity augmentation.
Embodiment AK: The method of any of Embodiments AA to AJ, wherein the second trained ML model comprises a plurality of convolutional neural network (CNN) layers.
Embodiment AL: The method of any of Embodiments AA to AK further comprising generating the first training dataset by applying raw imaging data to an image reconstruction pipeline.
Embodiment AM: The method of Embodiment AL, further comprising adding simulated image corruption to the raw imaging data.
Embodiment AN: The method of Embodiment AM, wherein the image corruption comprises at least one of a noise and/or an aliasing artifact.
Embodiment AO: The method of either Embodiment AM or AN, wherein adding simulated image corruption comprises adding simulated noise data.
Embodiment AP: The method of any of Embodiments AM to AO, wherein adding simulated image corruption comprises acquiring MR frequency data at a sub-Nyquist rate to simulate an aliasing artifact.
Embodiment AQ: The method of any of Embodiments AA to AP, wherein the third ML model is derived from the second ML model.
Embodiment AR: A device or system capable of performing any of the methods of Embodiments AA to AQ.
Embodiment BA: A method comprising acquiring a patient image using an imaging system, and applying a denoising and dealiasing machine-learning (ML) model to the patient image to obtain a denoised and/or dealiased patient image, the denoising and dealiasing ML model obtained by: (1) training a first ML model using a first training dataset comprising first image data to obtain a second ML model; and (2) training (a) the second ML model or (b) a third ML model using a second training dataset to obtain a fourth ML model, wherein the second training dataset comprises (i) the first image data and (ii) training image data obtained by applying at least one of the second ML model or the third ML model to second image data, wherein the denoising and dealiasing ML model is either the fourth ML model or derived from the fourth ML model.
Embodiment BB: The method of Embodiment BA, wherein the patient image is acquired using at least one of a low-field MR imaging system or a POC MR imaging system.
Embodiment BC: A device or system capable of performing either Embodiment BA or BB.
Embodiment CA: A system comprising an imaging system configured to generate imaging data, and one or more processors configured to cause the imaging system to generate patient images, and apply a denoising and dealiasing ML model to the patient images to generate denoised and/or dealiased patient images, the denoising and dealiasing ML model obtained by: using a first training dataset comprising first image data to obtain a first ML model; and using a second training dataset to obtain a second ML model, wherein the second training dataset comprises (i) the first image data and (ii) training image data obtained by applying at least one of the first ML model or a third ML model to second image data, and wherein the denoising and dealiasing ML model is either the second ML model or derived from the second ML model.
Embodiment CB: The system of Embodiment CA to perform any of the methods disclosed herein, such as any of Embodiments AA to BB.
Embodiment DA: A device or system to perform any of the methods disclosed herein, such as any of Embodiments AA to BB.
Embodiment EA: A method of generating a trained machine-learning (ML) model for image reconstruction, wherein generating the trained ML model comprises: (1) using a first training dataset to update a first ML model to obtain a second ML model, the first training dataset comprising first image data; and (2) using a second training dataset to update the second ML model to obtain the trained ML model, wherein the second training dataset comprises: (i) the first image data, and (ii) training image data obtained by applying the second ML model to second image data.
Embodiment EB: The method of Embodiment EA, wherein at least one of the first training dataset or the second training dataset comprises simulated imaging data.
Embodiment EC: The method of Embodiment EB, wherein the simulated imaging data is based on simulated images of arbitrary contrast.
Embodiment ED: The method of any of Embodiments EA to EC, wherein the second image data comprises non-independent and non-identically distributed noise.
Embodiment EE: The method of any of Embodiments EA to ED, further comprising applying the trained ML model to a patient image to obtain a reconstructed patient image.
Embodiment EF: The method of Embodiment EE, wherein the patient image is acquired using at least one of a low-field magnetic resonance (MR) imaging system or a point-of-care (POC) MR imaging system.
Embodiment EG: The method of any of Embodiments EA to EF, wherein the first image data and the second image data belong to separate domains.
Embodiment EH: The method of any of Embodiments EA to EG, further comprising augmenting the training image data based on an augmentation process before step (2).
Embodiment EI: The method of any of Embodiments EA to EH, wherein the second trained ML model comprises a plurality of convolutional neural network (CNN) layers.
Embodiment EJ: The method of any of Embodiments EA to EI, further comprising generating the first training dataset by applying raw imaging data to an image reconstruction pipeline.
Embodiment EK: The method of Embodiment EJ, further comprising adding simulated image corruption to the raw imaging data.
Embodiment FA: A method comprising acquiring a patient image using an imaging system, and applying a trained machine-learning (ML) model to the patient image to obtain a reconstructed patient image, the trained ML model having been generated by any of the methods disclosed herein, such as any of the methods of Embodiments AA to AQ or EA to EK.
Embodiment FB: The method of Embodiment FA, wherein the patient image is acquired using a low-field MR imaging system.
Embodiment FC: The method of Embodiment FA or FB, wherein the patient image is acquired using a point-of-care (POC) MR imaging system.
Embodiment GA: A non-transitory computer-readable storage medium comprising instructions that, when executed by one or more processors of a computing system, cause the computing system to perform any of the methods disclosed herein, such as any of the methods of Embodiments AA to AQ or EA to EK.
Embodiment HA: A non-transitory computer-readable storage medium comprising instructions that, when executed by one or more processors of a computing system, cause the computing system to: acquire patient image data using an imaging system; and obtain a reconstructed patient image based on the patient image data, wherein obtaining the reconstructed patient image comprises applying a trained machine-learning (ML) model to the patient image data, the trained ML model having been generated by any of the methods disclosed herein, such as any of the methods of Embodiments AA to AQ or EA to EK.
Embodiment IA: A system comprising an imaging system configured to generate imaging data, and one or more processors configured to cause the imaging system to generate patient images, and apply a trained ML model to the patient images to generate reconstructed patient images, the trained ML model having been generated by any of the methods disclosed herein, such as any of the methods of Embodiments AA to AQ or EA to EK.

A computing system or computing device comprising the computer-readable storage media of either Embodiment GA or HA.

The implementations described herein have been described with reference to drawings. The drawings illustrate certain details of specific implementations that implement the systems, methods, and programs described herein. Describing the implementations with drawings should not be construed as imposing on the disclosure any limitations that may be present in the drawings.

It should be understood that no claim element herein is to be construed under the provisions of 35 U.S.C. § 112(f), unless the element is expressly recited using the phrase “means for.”

As used herein, the term “circuit” may include hardware structured to execute the functions described herein. In some implementations, each respective “circuit” may include machine-readable media for configuring the hardware to execute the functions described herein. The circuit may be embodied as one or more circuitry components including, but not limited to, processing circuitry, network interfaces, peripheral devices, input devices, output devices, sensors, etc. In some implementations, a circuit may take the form of one or more analog circuits, electronic circuits (e.g., integrated circuits (IC), discrete circuits, system on a chip (SOC) circuits), telecommunication circuits, hybrid circuits, and any other type of “circuit.” In this regard, the “circuit” may include any type of component for accomplishing or facilitating achievement of the operations described herein. In a non-limiting example, a circuit as described herein may include one or more transistors, logic gates (e.g., NAND, AND, NOR, OR, XOR, NOT, XNOR), resistors, multiplexers, registers, capacitors, inductors, diodes, wiring, and so on.

The “circuit” may also include one or more processors communicatively coupled to one or more memory or memory devices. In this regard, the one or more processors may execute instructions stored in the memory or may execute instructions otherwise accessible to the one or more processors. In some implementations, the one or more processors may be embodied in various ways. The one or more processors may be constructed in a manner sufficient to perform at least the operations described herein. In some implementations, the one or more processors may be shared by multiple circuits (e.g., circuit A and circuit B may comprise or otherwise share the same processor, which, in some example implementations, may execute instructions stored, or otherwise accessed, via different areas of memory). Alternatively or additionally, the one or more processors may be structured to perform or otherwise execute certain operations independent of one or more co-processors.

In other example implementations, two or more processors may be coupled via a bus to enable independent, parallel, pipelined, or multi-threaded instruction execution. Each processor may be implemented as one or more general-purpose processors, ASICs, FPGAs, GPUs, TPUs, digital signal processors (DSPs), or other suitable electronic data processing components structured to execute instructions provided by memory. The one or more processors may take the form of a single core processor, multi-core processor (e.g., a dual core processor, triple core processor, or quad core processor), microprocessor, etc. In some implementations, the one or more processors may be external to the apparatus, in a non-limiting example, the one or more processors may be a remote processor (e.g., a cloud-based processor). Alternatively or additionally, the one or more processors may be internal or local to the apparatus. In this regard, a given circuit or components thereof may be disposed locally (e.g., as part of a local server, a local computing system) or remotely (e.g., as part of a remote server such as a cloud based server). To that end, a “circuit” as described herein may include components that are distributed across one or more locations.

An exemplary system for implementing the overall system or portions of the implementations might include a general purpose computing devices in the form of computers, including a processing unit, a system memory, and a system bus that couples various system components including the system memory to the processing unit. Each memory device may include non-transient volatile storage media, non-volatile storage media, non-transitory storage media (e.g., one or more volatile or non-volatile memories), etc. In some implementations, the non-volatile media may take the form of ROM, flash memory (e.g., flash memory such as NAND, 3D NAND, NOR, 3D NOR), EEPROM, MRAM, magnetic storage, hard discs, optical discs, etc. In other implementations, the volatile storage media may take the form of RAM, TRAM, ZRAM, etc. Combinations of the above are also included within the scope of machine-readable media. In this regard, machine-executable instructions comprise, in a non-limiting example, instructions and data, which cause a general-purpose computer, special purpose computer, or special purpose processing machines to perform a certain function or group of functions. Each respective memory device may be operable to maintain or otherwise store information relating to the operations performed by one or more associated circuits, including processor instructions and related data (e.g., database components, object code components, script components), in accordance with the example implementations described herein.

It should also be noted that the term “input devices,” as described herein, may include any type of input device including, but not limited to, a keyboard, a keypad, a mouse, joystick, or other input devices performing a similar function. Comparatively, the term “output device,” as described herein, may include any type of output device including, but not limited to, a computer monitor, printer, facsimile machine, or other output devices performing a similar function.

It should be noted that although the diagrams herein may show a specific order and composition of method steps, it is understood that the order of these steps may differ from what is depicted. In a non-limiting example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative implementations. Accordingly, all such modifications are intended to be included within the scope of the present disclosure as defined in the appended claims. Such variations will depend on the machine-readable media and hardware systems chosen and on designer choice. It is understood that all such variations are within the scope of the disclosure. Likewise, software and web implementations of the present disclosure could be accomplished with standard programming techniques with rule-based logic and other logic to accomplish the various database searching steps, correlation steps, comparison steps, and decision steps.

While this specification contains many specific implementation details, these should not be construed as limitations on the scope of any inventions or of what may be claimed, but rather as descriptions of features specific to particular implementations of the systems and methods described herein. Certain features that are described in this specification in the context of separate implementations may also be implemented in combination in a single implementation. Conversely, various features that are described in the context of a single implementation may also be implemented in multiple implementations separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination.

In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products.

Having now described some illustrative implementations and implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements, and features discussed only in connection with one implementation are not intended to be excluded from a similar role in other implementations.

The phraseology and terminology used herein is for the purpose of description and should not be regarded as limiting. The use of “including,” “comprising,” “having,” “containing,” “involving,” “characterized by,” “characterized in that,” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any act or element being based on any information, act, or element may include implementations where the act or element is based at least in part on any information, act, or element.

Any implementation disclosed herein may be combined with any other implementation, and references to “an implementation,” “some implementations,” “an alternate implementation,” “various implementation,” “one implementation,” or the like are not necessarily mutually exclusive and are intended to indicate that a particular feature, structure, or characteristic described in connection with the implementation may be included in at least one implementation. Such terms as used herein are not necessarily all referring to the same implementation. Any implementation may be combined with any other implementation, inclusively or exclusively, in any manner consistent with the aspects and implementations disclosed herein.

References to “or” may be construed as inclusive so that any terms described using “or” may indicate any of a single, more than one, and all of the described terms.

Where technical features in the drawings, detailed description or any claim are followed by reference signs, the reference signs have been included for the sole purpose of increasing the intelligibility of the drawings, detailed description, and claims. Accordingly, neither the reference signs nor their absence have any limiting effect on the scope of any claim elements.

The foregoing description of implementations has been presented for purposes of illustration and description. It is not intended to be exhaustive or to limit the disclosure to the precise form disclosed, and modifications and variations are possible in light of the above teachings or may be acquired from this disclosure. The implementations were chosen and described in order to explain the principals of the disclosure and its practical application to enable one skilled in the art to utilize the various implementations and with various modifications as are suited to the particular use contemplated. Other substitutions, modifications, changes, and omissions may be made in the design, operating conditions and implementation of the implementations without departing from the scope of the present disclosure as expressed in the appended claims.

Example, non-limiting aspects and features may include any combination of one or more of the following:
Example A1: A method, system comprising one or more processors, and/or non-transitory computer-readable storage medium comprising instructions executable by the one or more processors, wherein the second image data comprises non-independent and non-identically distributed noise, and wherein step (2) comprises re-training the second ML model using the second training dataset that includes pseudo-clean images generated by applying the second ML model to the second image data.
Example A2: A method, system comprising one or more processors, and/or non-transitory computer-readable storage medium comprising instructions executable by the one or more processors, wherein generating the second training dataset comprises applying at least one of image sharpening, affine transformation, elastic deformation, insertion of geometric objects, or intensity augmentation to the training image data obtained by applying the second ML model to the second image data.
Example A3: A method, system comprising one or more processors, and/or non-transitory computer-readable storage medium comprising instructions executable by the one or more processors, wherein the trained ML model comprises a convolutional neural network including at least one discrete wavelet transform (DWT) layer and at least one inverse discrete wavelet transform (IDWT) layer, and is configured to process multi-slice magnetic resonance image data to generate a denoised and/or dealiased image corresponding to a middle slice of the multi-slice input.
Example A4: A method, system comprising one or more processors, and/or non-transitory computer-readable storage medium comprising instructions executable by the one or more processors, wherein the trained ML model comprises at least one data consistency layer configured to enforce consistency between reconstructed image data and acquired MR spatial frequency data, the data consistency layer being implemented using a conjugate gradient descent algorithm and incorporating at least one of sample density compensation or spectral normalization.
Example A5: A method, system comprising one or more processors, and/or non-transitory computer-readable storage medium comprising instructions executable by the one or more processors, wherein the patient image comprises a diffusion-weighted magnetic resonance image acquired at a magnetic field strength of less than 0.1 Tesla and at a b-value between 800 s/mm² and 1000 s/mm².
Example A6: A method, system comprising one or more processors, and/or non-transitory computer-readable storage medium comprising instructions executable by the one or more processors, wherein at least one of the first training dataset or the second training dataset comprises simulated magnetic resonance images generated using a Bloch equation simulation or MR contrast equation to produce images of arbitrary contrast.
Example A7: A method, system comprising one or more processors, and/or non-transitory computer-readable storage medium comprising instructions executable by the one or more processors, wherein generating the first training dataset comprises replacing at least a portion of an MR image with content from a natural image or inserting geometric objects into anatomical structures of the MR image.
Example A8: A method, system comprising one or more processors, and/or non-transitory computer-readable storage medium comprising instructions executable by the one or more processors, wherein the image reconstruction pipeline comprises transforming non-Cartesian MR spatial frequency data to the image domain using a gridding operation with sample density compensation prior to applying the trained ML model.
Example A9: A method, system comprising one or more processors, and/or non-transitory computer-readable storage medium comprising instructions executable by the one or more processors, further comprising generating the reconstructed patient image by combining outputs of a plurality of trained ML models using an ensemble operation.
Example A10: A method, system comprising one or more processors, and/or non-transitory computer-readable storage medium comprising instructions executable by the one or more processors, wherein the simulated image corruption comprises simulating patient motion by multiplying MR spatial frequency data by complex exponentials, and wherein the trained ML model is configured to correct motion artifacts in the reconstructed patient image.

What is claimed is:

1. A method of generating a trained machine-learning (ML) model for image reconstruction, wherein generating the trained ML model comprises:
(1) using a first training dataset to update a first ML model to obtain a second ML model, the first training dataset comprising first image data; and
(2) using a second training dataset to update the second ML model to obtain the trained ML model, wherein the second training dataset comprises:
(i) the first image data, and
(ii) training image data obtained by applying the second ML model to second image data.

2. The method of claim 1, wherein at least one of the first training dataset or the second training dataset comprises simulated imaging data.

3. The method of claim 2, wherein the simulated imaging data is based on simulated images of arbitrary contrast.

4. The method of claim 1, wherein the second image data comprises non-independent and non-identically distributed noise.

5. The method of claim 1, further comprising applying the trained ML model to a patient image to obtain a reconstructed patient image.

6. The method of claim 5, wherein the patient image is acquired using at least one of a low-field magnetic resonance (MR) imaging system or a point-of-care (POC) MR imaging system.

7. The method of claim 1, wherein the first image data and the second image data belong to separate domains.

8. The method of claim 1, further comprising augmenting the training image data based on an augmentation process before step (2).

9. The method of claim 1, wherein the second trained ML model comprises a plurality of convolutional neural network (CNN) layers.

10. The method of claim 1, further comprising generating the first training dataset by applying raw imaging data to an image reconstruction pipeline.

11. The method of claim 10, further comprising adding simulated image corruption to the raw imaging data.

12. A method comprising acquiring a patient image using an imaging system, and applying a trained machine-learning (ML) model to the patient image to obtain a reconstructed patient image, the trained ML model having been generated by the method of claim 1.

13. The method of claim 12, wherein the patient image is acquired using at least one of a low-field MR imaging system or a POC MR imaging system.

14. A non-transitory computer-readable storage medium comprising instructions that, when executed by one or more processors of a computing system, cause the computing system to:
acquire patient image data using an imaging system; and
obtain a reconstructed patient image based on the patient image data, wherein obtaining the reconstructed patient image comprises applying a trained machine-learning (ML) model to the patient image data, the trained ML model having been generated by the method of claim 1.

15. A system comprising an imaging system configured to generate imaging data, and one or more processors configured to cause the imaging system to generate patient images, and apply a trained ML model to the patient images to generate reconstructed patient images, the trained ML model having been generated by the method of claim 1.
